SEQUENCE LISTING (8 sequences)

SEQ ID NO: 1 - protein, 9 aa, Artificial Sequence
  Description of Artificial Sequence note = synthetic construct
LYLVCGERG

SEQ ID NO: 2 - protein, 220 aa, Artificial Sequence
  Description of Artificial Sequence note = synthetic construct
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 3 - DNA, 3661 bp, Artificial Sequence
  Description of Artificial Sequence note = synthetic construct
agcaaacacatccgtagaaggcagcgcggccgccgagagccgcagcgccgctcgcccgccgccccccaccccgccgccccgcccggcgaattgcgccccgcgcccctcccctcgcgcccccgagacaaagaggagagaaagtttgcgcggccgagcggggcaggtgaggagggtgagccgcgcgggaggggcccgcctcggccccggctcagcccccgcccgcgcccccagcccgccgccgcgagcagcgcccggaccccccagcggcggcccccgcccgcccagccccccggcccgccatgggcgccgcggcccgcaccctgcggctggcgctcggcctcctgctgctggcgacgctgcttcgcccggccgacgcctgcagctgctccccggtgcacccgcaacaggcgttttgcaatgcagatgtagtgatcagggccaaagcggtcagtgagaaggaagtggactctggaaacgacatttatggcaaccctatcaagaggatccagtatgagatcaagcagataaagatgttcaaagggcctgagaaggatatagagtttatctacacggccccctcctcggcagtgtgtggggtctcgctggacgttggaggaaagaaggaatatctcattgcaggaaaggccgagggggacggcaagatgcacatcaccctctgtgacttcatcgtgccctgggacaccctgagcaccacccagaagaagagcctgaaccacaggtaccagatgggctgcgagtgcaagatcacgcgctgccccatgatcccgtgctacatctcctccccggacgagtgcctctggatggactgggtcacagagaagaacatcaacgggcaccaggccaagttcttcgcctgcatcaagagaagtgacggctcctgtgcgtggtaccgcggcgcggcgccccccaagcaggagtttctcgacatcgaggacccataagcaggcctccaacgcccctgtggccaactgcaaaaaaagcctccaagggtttcgactggtccagctctgacatcccttcctggaaacagcatgaataaaacactcatcccatgggtccaaattaatatgattctgctccccccttctccttttagacatggttgtgggtctggagggagacgtgggtccaaggtcctcatcccatcctccctctgccaggcactatgtgtctggggcttcgatccttgggtgcaggcagggctgggacacgcggcttccctcccagtccctgccttggcaccgtcacagatgccaagcaggcagcacttagggatctcccagctgggttagggcagggcctggaaatgtgcattttgcagaaacttttgagggtcgttgcaagactgtgtagcaggcctaccaggtccctttcatcttgagagggacatggcccttgttttctgcagcttccacgcctctgcactccctgcccctggcaagtgctcccatcgccccggtgcccaccatgagctcccagcacctgactccccccacatccaagggcagcctggaaccagtggctagttcttgaaggagccccatcaatcctattaatcctcagaattccagtgggagcctccctctgagccttgtagaaatgggagcgagaaaccccagctgagctgcgttccagcctcagctgagtctttttggtctgcacccacccccccaccccccccccgcccacatgctccccagcttgcaggaggaatcggtgaggtcctgtcctgaggctgctgtccggggccggtggctgccctcaaggtcccttccctagctgctgcggttgccattgcttcttgcctgttctggcatcaggcacctggattgagttgcacagctttgctttatccgggcttgtgtgcagggcccggctgggctccccatctgcacatcctgaggacagaaaaagctgggtcttgctgtgccctcccaggcttagtgttccctccctcaaagactgacagccatcgttctgcacggggttttctgcatgtgacgccagctaagcatagtaagaagtccagcctaggaagggaaggattttggaggtaggtggctttggtgacacactcacttctttctcagcctccaggacactatggcctgttttaagagacatcttatttttctaaaggtgaattctcagatgataggtgaacctgagttgcagatataccaacttctgcttgtatttcttaaatgacaaagattacctagctaagaaacttcctagggaactagggaacctatgtgttccctcagtgtggtttcctgaagccagtgatatgggggttaggataggaagaactttctcggtaatgataaggagaatctcttgtttcctcccacctgtgttgtaaagataaactgacgatatacaggcacattatgtaaacatacacacgcaatgaaaccgaagcttggcggcctgggcgtggtcttgcaaaatgcttccaaagccaccttagcctgttctattcagcggcaaccccaaagcacctgttaagactcctgacccccaagtggcatgcagcccccatgcccaccgggacctggtcagcacagatcttgatgacttccctttctagggcagactgggagggtatccaggaatcggcccctgccccacgggcgttttcatgctgtacagtgacctaaagttggtaagatgtcataatggaccagtccatgtgatttcagtatatacaactccaccagacccctccaacccatataacaccccacccctgttcgcttcctgtatggtgatatcatatgtaacatttactcctgtttctgctgattgtttttttaatgttttggtttgtttttgacatcagctgtaatcattcctgtgctgtgttttttattacccttggtaggtattagacttgcacttttttaaaaaaaggtttctgcatcgtggaagcatttgacccagagtggaacgcgtggcctatgcaggtggattccttcaggtctttcctttggttctttgagcatctttgctttcattcgtctcccgtctttggttctccagttcaaattattgcaaagtaaaggatctttgagtaggttcggtctgaaaggtgtggcctttatatttgatccacacacgttggtcttttaaccgtgctgagcagaaaacaaaacaggttaagaagagccgggtggcagctgacagaggaagccgctcaaataccttcacaataaatagtggcaatatatatatagtttaagaaggctctccatttggcatcgtttaatttatatgttatgttctaagcacagctctcttctcctattttcatcctgcaagcaactcaaaatatttaaaataaagtttacattgtagttattttcaaatctttgcttgataagtattaagaaatattggacttgctgccgtaatttaaagctctgttgattttgtttccgtttggatttttgggggaggggagcactgtgtttatgctggaatatgaagtctgagaccttccggtgctgggaacacacaagagttgttgaaagttgacaagcagactgcgcatgtctctgatgctttgtatcattcttgagcaatcgctcggtccgtggacaataaacagtattatcaaagagaaaaaaaaaaaaaa

SEQ ID NO: 4 - protein, 211 aa, Artificial Sequence
  Description of Artificial Sequence note = synthetic construct
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 5 - DNA, 1020 bp, Artificial Sequence
  Description of Artificial Sequence note = synthetic construct
cccgccggcggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaaaa

SEQ ID NO: 6 - DNA, 1 bp, Artificial Sequence
  Description of Artificial Sequence note = synthetic construct
a

SEQ ID NO: 7 - protein, 224 aa, Artificial Sequence
  Description of Artificial Sequence note = synthetic construct
MPGSPRPAPSWVLLLRLLALLRPPGLGEACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 8 - DNA, 1463 bp, Artificial Sequence
  Description of Artificial Sequence note = synthetic construct
atcccctctcccagtgcttcccctctgcttccagatcgcttcatgacttaggcagggaaacagaggtcagggcctccttccaggcttccctctgcatcttactgagtatgcaggtcggaagagcctcgggtcctgcctccgcgggtggcctagagccaaaggaaggcggagcccgtcggggcgggattggcccttagggccacctcataaagcctggggcgaggggcacaacggccttgggaaggagccctgctggggccgtccagtcccccagacctcacaggctcagtcgcggatctgcagtgtcatgcctgggagccctcggcccgcgccaagctgggtgctgttgctgcggctgctggcgttgctgcggcccccggggctgggtgaggcatgcagctgcgccccggcgcaccctcagcagcacatctgccactcggcacttgtgattcgggccaaaatctccagtgagaaggtagttccggccagtgcagaccctgctgacactgaaaaaatgctccggtatgaaatcaaacagataaagatgttcaaagggtttgagaaagtcaaggatgttcagtatatctatacgccttttgactcttccctctgtggtgtgaaactagaagccaacagccagaagcagtatctcttgactggtcaggtcctcagtgatggaaaagtcttcatccatctgtgcaactacatcgagccctgggaggacctgtccttggtgcagagggaaagtctgaatcatcactaccatctgaactgtggctgccaaatcaccacctgctacacagtaccctgtaccatctcggcccctaacgagtgcctctggacagactggctgttggaacgaaagctctatggttaccaggctcagcattatgtctgtatgaagcatgttgacggcacctgcagctggtaccggggccacctgcctctcaggaaggagtttgttgacatcgttcagccctagtagggaccagtgaccatcacatcccttcaagagtcctgaagatcaagccagttctccttccctgcagagctttggccattaccacctgacctcttgctgccagctaataagaagtgccaagtggacagtctggccactgtcaaggcagggaaggggccatgacttttctgccctgccctcagcctgttgcccctgcctcccaaaccccattagtctagccttgtagctgttactgcaagtgtttcttctggcttagtctgttttctaaagccaggactattccctttcctccccaggaatatgtgttttcctttgtcttaatcgatctggtaggggagaaatggcgaatgtcatacacatgagatggtatatccttgcgatgtacagtatcagaaggtggtttgacagcatcataaacaggctgactggcaggaatgaaaacaagaaaaaaaaaaaaaaaaaaaaaaaaaaaaaaaaaaaa

(19) United States
(12) Patent Application Publication
(10) Pub. No.: US 2007/0203244 A1
(43) Pub. Date: Aug. 30, 2007

(54) INHIBITORS OF MEMBRANE TYPE-1 MATRIX METALLOPROTEINASE FOR THE TREATMENT OF INSULIN-DEPENDENT DIABETES MELLITUS

(76) Inventors: Alex Y. Strongin, LaJolla, CA (US); Alexei Y. Savinov, LaJolla, CA (US); Dimitri V. Rozanov, LaJolla, CA (US)

Correspondence Address: NEEDLE & ROSENBERG, P.C., SUITE 1000, 999 PEACHTREE STREET, ATLANTA, GA, 30309-3915, US

(21) Appl. No.: 11/672,405

(22) Filed: Feb. 7, 2007

Related U.S. Application Data
(60) Provisional application No. 60/772,058, filed on Feb. 9, 2006.

(51) Int. Cl. A61K 31/19

(52) U.S. Cl. 514/575

(57) ABSTRACT

Provided herein are compositions and methods for inhibiting the transmigration of T cells through pancreatic capillary endothelium and treating insulin-dependent diabetes mellitus (IDDM; type I diabetes) using inhibitors of MT1-MMP.

### CROSS-REFERENCE TO RELATED APPLICATIONS

This application claims benefit of U.S. Provisional Application No. 60/772,058, filed Feb. 9, 2006, which is hereby incorporated herein by reference in its entirety.

### STATEMENT REGARDING FEDERALLY SPONSORED RESEARCH

This invention was made with government support under Grant CA83017, CA77470 and RR020843 awarded by National Institutes of Health. The government has certain rights in the invention.

### BACKGROUND OF THE INVENTION

Insulin-dependent diabetes mellitus (IDDM; type I diabetes) is a major, debilitating, T cell-mediated, autoimmune disease (Homann, D. & von Herrath, M. (2004)). The pathogenesis of IDDM involves the activation of autoimmune T cells followed by their homing into the pancreatic islets. In the islets, T cells directly destroy insulin-producing β cells (Mathis, D., et al. (2001)). The cell-surface adhesion receptor CD44 is elevated in activated T cells. CD44, via its interactions with endothelial hyaluronan, mediates T cell adhesion on the endothelium and the subsequent transmigration events (DeGrendele, H. C., et al. (1997)).

There is evidence that CD44 is a target of MT1-MMP proteolysis in tumor cells. MT1-MMP cleavage releases the extracellular domain of CD44 from cell surfaces and inactivates the CD44 cell receptor function (Mori, H., et al. (2002); Nakamura, H., et al. (2004); Suenaga, N., et al. (2005)). Invasion-promoting MT1-MMP, a multifunctional membrane-tethered enzyme, functions in cancer cells as one of the main mediators of pericellular proteolytic events, and directly cleaves cell surface receptors (Egeblad, M. & Werb, Z. (2002); Sabeh, F., et al. (2004); Seiki, M. (2003)). Provided herein are compositions and methods for the treatment of IDDM using inhibitors of MT1-MMP.

### BRIEF SUMMARY OF THE INVENTION

Provided herein are methods of inhibiting the transmigration of T cells through pancreatic capillary endothelium, comprising administering to the cells a composition comprising an inhibitor of membrane type matrix metalloproteinase (MT-MMP).

Also provided herein are methods of treating type I diabetes in a subject, comprising administering to the subject a composition comprising an inhibitor of MT-MMP.

Also provided are methods of identifying a molecule, comprising screening a candidate molecule for the ability to inhibit MT-MMP activity, and determining if the candidate molecule can inhibit the transmigration of T cells through pancreatic capillary endothelium.

Also provided are methods of identifying a molecule, comprising determining if a molecule that inhibits MT-MMP activity can inhibit the transmigration of T cells through pancreatic capillary endothelium.

Also provided are methods of immobilizing T cells on pancreatic capillary endothelium, comprising contacting the cells with a composition comprising an inhibitor of MT-MMP. The cells of the method can be in or from a subject identified as a subject in need of immobilization of T cells on pancreatic capillary endothelium.

Also provided are methods of treating a subject at risk of type I diabetes, comprising administering to the subject a composition comprising an inhibitor of MT-MMP.

Additional advantages of the disclosed methods and compositions will be set forth in part in the description which follows, and in part will be understood from the description, or can be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows that excessive MT1-MMP proteolysis of CD44 decreases the rate of islet homing of T cells and delays the onset of diabetes in mice. FIG. 1A shows FACS analyses of IS-CD8⁺ cells. IS-CD8⁺ cells were stained with the MT1-MMP and CD44 antibodies, followed by the fluorescein isothiocyanate-conjugated secondary antibody, and then subjected to FACS analyses. Similar results were obtained when CD44 was stained with soluble fluorescently labeled hyaluronan. Left panel, staining of MT1-MMP (bold line, MT1-MMP; dotted line, isotype control). Right panel, staining of CD44 (bold line, untreated cells; dotted line, cells co-incubated with MT1-MMP-CAT). FIG. 1B shows MT1-MMP sheds cellular CD44 and releases its soluble fragments into the medium. IS-CD8⁺ cells were surface biotinylated and then co-incubated with MT1-MMP-CAT. The cells were then lysed with N-octyl-β-Dglucopyranoside supplemented with a protease inhibitor mixture. Biotin-labeled CD44 was captured from the cell lysate, and the captured samples were examined by Western blotting with the CD44 antibody to determine the released, soluble, CD44 ectodomain (Medium) and the residual, membrane-anchored CD44 (Cells). Where indicated, GM6001 was added to the samples. FIG. 1C shows MT1-MMP proteolysis of CD44 reduces diabetogenicity of IS-CD8⁺ cells. Left panel, cells were co-incubated with MT1-MMP-CAT, labeled with a fluorescent DiI dye, and then injected into NOD mice. In 24 h, the number of the labeled cells within the islets was counted in the cryostat sections of the pancreas. Right panel, MT1-MMP-CAT-treated and untreated IS-CD8⁺ cells were injected into NOD mice. The incidence of diabetes was 100% (6 of 6) with untreated cells and 70% (7 of 10) with the cells co-incubated with MT1-MMP-CAT.

FIG. 2 shows that proteolytically active MT1-MMP activates MMP-2 and cleaves cellular CD44 in adherent IS-CD8⁺ cells. IS-CD8⁺ cells were either allowed to adhere to plastic coated with gelatin or kept in solution. Top panel, cells adherent to gelatin-coated plastic (A) and non-adherent cells in suspension (NA) were co-incubated with purified MMP-2 (MMP-2 alone; no cells). In 4 h, media samples were withdrawn and analyzed by gelatin zymography to identify the proteolytic activity and the activation status of MMP-2. To observe the activation of MMP-2 naturally synthesized by T cells, no external MMP-2 was added to the two samples on the left. P, I, and E, the 68-kDa pro-enzyme, the 64-kDa intermediate, and the active 62-kDa mature enzyme of MMP-2. Bottom panel, cells were either surface-biotinylated and allowed to adhere to gelatin or kept in suspension. Cell lysate and medium aliquots were captured with streptavidin-agarose beads. CD44 was analyzed in the captured samples by Western blotting with a CD44 antibody.

FIG. 3 shows that a hydroxamate inhibitor, AG3340, inactivates MT1-MMP, blocks CD44 shedding in T cells, and delays the onset of transferred diabetes in NOD mice. FIG. 3A shows AG3340 delays the onset of adoptively transferred diabetes in NOD mice. IS-CD8⁺ cells and the splenocytes were each injected intravenously into NOD mice (1×10⁷ and 1.5×10⁷ cells/mouse, respectively; 6 mice/group). On day 0, 2, 4, 6, 8, and 10 following injection of the cells, mice received intraperitoneal injection with AG3340 (30 and 1 mg/kg). FIG. 3B shows AG3340 inhibits the transmigration of IS-CD8⁺ cells into the pancreatic islets. IS-CD8⁺ cells were co-incubated for 2 h with and without AG3340 (50 μM or 21 μg/ml) and then labeled with DiI. The labeled cells were next injected intravenously into NOD mice. In 24 h, the labeled cells at the entrance of the islet and within the pancreatic islets were each counted in the cryostat sections of the entire pancreas. n, total number of islets in each experimental group. FIG. 3C shows representative images of the pancreatic islets from NOD mice that received injection with DiI-labeled IS-CD8⁺ cells. Images were taken 24 h after injection. Dotted line surrounds the islet. White solid line indicates the islet-relevant area, within which the DiI-labeled cells were counted. Bottom panel, cells were pre-incubated with AG3340; top panel, intact cells. Note that AG3340 blocks the entrance of IS-CD8⁺ cells into the islet. FIG. 3D shows MT1-MMP proteolysis dynamically regulates the functionality of T cell CD44 in diapedesis. Low levels of MT1-MMP stimulate adhesion of T cells to the hyaluronan-rich endothelium. After T cell adhesion, T cell MT1-MMP is activated. High levels of MT1-MMP activity cause a CD44 deficiency. This event stimulates the transendothelial migration of T cells. Persistent CD44 excess reduces T cell homing and diapedesis.

FIG. 4 shows CD44 plays a major role in the homing of diabetogenic IS-CD8+ T cells to the pancreatic islets. The function-blocking antibody IM7.8.1 against CD44 and AG3340 were each injected i.v. in NOD mice. In 30 min, this injection was followed by the i.v. injection of DiI-labeled IS-CD8+ T cells. After 24 h, the labeled cells at the entrance of the islet and within the pancreatic islets were each counted in the cryostat sections of the pancreas using a fluorescent microscope. At least 100 islets per mouse (4-5 mice/group) were examined. The results are summarized in the left panel. * and **, p<0.05 by Fisher's test. Representative sections show the efficient homing of T cells in untreated animals, the drastic inhibitory effect of the function-blocking CD44 antibody and the AG3340-induced immobilization of T cells at the entrance of the islet.

FIG. 5 shows that inhibitory analysis confirms that intrinsic MT1-MMP cleaves cell-surface CD44 in IS-CD8+ T cells. Upper panels, cells were surface-biotinylated and then allowed to adhere in serum-free medium to plastic coated with I collagen/gelatin (adherent, A) or were kept in suspension (non-adherent, NA). Where indicated, TIMP-1 (100 ng/ml; a poor inhibitor of MT1-MMP), and TIMP-2 (100 ng/ml) and AG3340 (50 μM), both of which are highly potent inhibitors of MT1-MMP, were added to the cells. Cell lysate and medium aliquots were captured with streptavidin-agarose beads. CD44 was analyzed in the captured samples by Western blotting with an antibody to the CD44 ectodomain. Bottom panel shows that, to analyze MMP-2, adherent and non-adherent cells were each incubated for 18 h in serum-free medium. Purified MMP-2 (20 ng; MMP-2 alone; no cells) was added to the cells. The activation of MMP-2 was analyzed by gelatin zymography of the medium aliquots. No external MMP-2 was used in the Western blotting experiments (two upper panels).

FIG. 6 shows AG3340 reduces insulitis and stimulates regeneration of the islets in NOD mice with spontaneous diabetes. After development of spontaneous diabetes, insulin (15-20 U/kg; one injection in every two-three days) was injected s.c. in mice. Control animals (6 mice/group) received insulin alone, while an experimental group (5 mice/group) received insulin s.c. jointly with AG3340 i.p. Injections were continued for 40 days and then mice were sacrificed. Leukocytes and granulated β cells were stained with H&E and aldehyde fuchsin, respectively, in the sections of pancreata. The severity of insulitis of the islets (≧100/mouse) was scored (0, no lesions; 1, peri-insular leukocytic aggregates and, in addition, periductal infiltrates; 2, <25% islet destruction; 3, >25% islet destruction; and 4, totally destroyed islets). * and **, p=0.042 and p=0.037 by Fisher's test, respectively. Representative images of pancreatic sections of the control and AG3340-treated mice stained with an insulin antibody. Note islet mononuclear infiltration and extensive insulitis in the control, and the limited peri-insulitis as well as the formation of small, regenerating, insulin-positive islets in mice which received AG3340.

FIG. 7 shows AG3340 inhibits MT1-MMP and the shedding of CD44 in IS-CD8+ T cells. The upper panel shows gelatin zymography of MMP-2. To analyze the activation of MMP-2 by cellular MT1-MMP, adherent and non-adherent cells were each incubated for 18 h in serum-free medium. Purified MMP-2 (20 ng) was added to the cells. The activation of MMP-2 was analyzed by gelatin zymography of the medium aliquots to observe the conversion of the 68 kDa MMP-2 proenzyme into the 62 kDa MMP-2 mature enzyme. Where indicated, AG3340, SB-3CT and EGCG were added to the cells for 18 h. The middle panel shows Western blotting of CD44. Cells were surface-biotinylated and then allowed to adhere, in serum-free medium, to plastic coated with I collagen/gelatin (adherent, A) or remained in suspension (non-adherent, NA). Where indicated, AG3340, SB-3CT and EGCG were added to the cells. Cell lysate and medium samples were captured with streptavidin-agarose beads. CD44 was analyzed in the captured sample aliquots (50 μg total protein each) by Western blotting with an antibody to the CD44 ectodomain. The bottom panel shows MMP-2 is inhibited by low concentrations of SB-3CT. α1-Antitrypsin was incubated with MMP-2. The digest samples were analyzed by reducing SDS-gel electrophoresis. Where indicated, SB-3CT was added to the samples in the concentrations shown on the figure.

FIG. 8 shows AG3340 inhibits the intra-islet homing of IS-CD8⁺ T cells. AG3340, SB-3CT and EGCG were each injected in NOD mice. In 30 min, each injection was followed by the injection of DiI-labeled IS-CD8⁺ T cells. After 24 h, the cryostat sections of the pancreata were examined with a fluorescence microscope. The DiI-labeled cells were ascribed their position either at the entrance of the islet or inside the pancreatic islets and counted. At least 100 islets per mouse (4-5 mice/group) were examined. The islets are easily recognized by their morphological characteristics including lower fluorescence and a compact, dense, structure. Representative images of the pancreatic islets from NOD mice that received an injection with DiI-labeled cells are shown.

FIG. 9 shows AG3340 inhibits transendothelial migration of IS-CD8⁺ T cells and delays the onset of transferred diabetes in NOD mice. FIG. 9A shows AG3340 inhibits the transmigration of IS-CD8⁺ cells into the pancreatic islets. Mice received either AG3340, SB-3CT, EGCG or PBS 30 min prior to the injection of the cells. IS-CD8⁺ cells were labeled with DiI and then injected in NOD mice. In 24 h, the labeled cells with their intra-islet location were counted in the cryostat sections of the entire pancreas. FIG. 9B shows AG3340 delays the onset of adoptively transferred diabetes in NOD mice. IS-CD8⁺ cells were injected in NOD mice. Mice received either AG3340, SB-3CT and EGCG or PBS one injection every other day until they developed diabetes (approximately 1-2 weeks). Stock solutions of SB-3CT (60 mg/ml), EGCG (50 mg/ml) and AG3340 (50 mg/ml) were made in 50% DMSO. Immediately prior to injections, EGCG and SB-3CT were each diluted in PBS to a concentration of 4 mg/ml. AG3340 was diluted in PBS to reach a concentration of 0.4 mg/ml. PBS containing 3.% DMSO was used as a vehicle control. The onset of diabetes was monitored daily by measuring urine glucose levels with the Diastix reagent strips. Mice with urine glucose levels of ≧2000 mg/dl for 3 consecutive days were considered diabetic. * and **, p=0.02 and p=0.015 by Fisher's test, respectively.

### DETAILED DESCRIPTION OF THE INVENTION

Provided herein are compositions and methods relating to inhibitors of membrane type matrix metalloproteinase (MT-MMP).

The disclosed methods and compositions can be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### A. MATERIALS

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

1. Inhibitors

The inhibitor of the herein provided methods can be a native tissue inhibitor of MMP (TIMP). The TIMP can be TIMP-2. The TIMP can be TIMP-3. The TIMP can be TIMP-4. A review of TIMPs can be found in the Dissertation of Palosaari, H (Acta Universitatis Ouluensis Medica, D 739, ISBN 951-42-7077-0), which is hereby incorporated by reference in its entirety for this teaching.

A feature of all TIMPs is that they have 12 conserved cysteine residues, with conserved relative spacing, and the presence of a 23 to 29 amino acid leader sequence, which is cleaved to produce a mature protein. Crystal structures for TIMPs, and MMP-TIMP complexes such as TIMP-1 in complex with MMP-3 and TIMP-2 with MT1-MMP have been described (Gomis-Ruth et al. 1997, Fernandez-Catalan et al. 1998). TIMPs have the shape of an elongated, contiguous wedge consisting of the N-terminal and the C-terminal halves of the polypeptide chains opposing each other (Gomis-Ruth et al. 1997). In complexes with MMPs, TIMPs bind with their edge into the entire length of the active-site cleft of MMPs (Fernandez-Catalan et al. 1998, Gomis-Ruth et al. 1997).

TIMP-2 is a nonglycosylated protein of 21 kDa molecular mass (Stetler-Stevenson et al. 1989a, Boone et al. 1990). It has an extended negatively charged C-terminus (Boone et al. 1990). The TIMP-2 promoter contains several regulatory elements including five Sp1, two AP-2, one AP-1 and three PEA-3 binding sites (De Clerck et al. 1994, Hammani et al. 1996). TIMP-2 is transcribed into two mRNAs of 1.2 and 3.8 kb (Hammani et al. 1996).

Human TIMP-2 comprises the amino acid sequence set forth in SEQ ID NO:2 and is encoded by the nucleic acid sequence set forth in SEQ ID NO:3 (Accession No. BC071586). Thus, the inhibitor of the provided methods can comprise the amino acid sequence set forth in SEQ ID NO:2, or a biologically active fragment thereof. The inhibitor can also comprise an amino acid having at least 70%, 75%, 80%, 85%, 90%, 95% homology to the amino acid sequence set forth in SEQ ID NO:2, or a biologically active fragment thereof. The inhibitor of the provided methods can also comprise a nucleic acid encoding the amino acid sequence set forth in SEQ ID NO:2, or a biologically active fragment thereof. Thus, the inhibitor of the provided methods can comprise the nucleic acid sequence set forth in SEQ ID NO:3. The inhibitor can also comprise nucleic acid having at least 70%, 75%, 80%, 85%, 90%, 95% sequence identity to the nucleic acid sequence set forth in SEQ ID NO:3, wherein the nucleic acid comprises at least 20, 30, 40, 50, 100 nucleotides.

The TIMP-3 polypeptide sequence is 37% and 42% similar to the sequences of TIMP-1 and TIMP-2, respectively (Apte et al. 1994). It has a conserved glycosylation site near the C-terminus. Characterization of the human recombinant TIMP-3 reveals that it has both a 27 kDa glycosylated and a 24 kDa unglycosylated species (Apte et al. 1995). TIMP-3 is localized to the ECM in both its glycosylated and unglycosylated forms (Langton et al. 1998). The TIMP-3 gene has four Sp1 sites, but no TATA-box in the promoter (Apte et al. 1994, Wick et al. 1995). Three TIMP-3 mRNA species of 2.4, 2.8 and 5.5 kb are transcribed from the gene (Apte et al. 1994), and are constitutively expressed by human chondrocytes (Su et al. 1996).

Human TIMP-3 comprises the amino acid sequence set forth in SEQ ID NO:4 and is encoded by the nucleic acid sequence set forth in SEQ ID NO:5 (Accession No. X76227). Thus, the inhibitor of the provided methods can comprise the amino acid sequence set forth in SEQ ID NO:4, or a biologically active fragment thereof. The inhibitor can also comprise an amino acid having at least 70%, 75%, 80%, 85%, 90%, 95% homology to the amino acid sequence set forth in SEQ ID NO:4, or a biologically active fragment thereof. The inhibitor of the provided methods can also comprise a nucleic acid encoding the amino acid sequence set forth in SEQ ID NO:4, or a biologically active fragment thereof. Thus, the inhibitor of the provided methods can comprise the nucleic acid sequence set forth in SEQ ID NO:5. The inhibitor can also comprise nucleic acid having at least 70%, 75%, 80%, 85%, 90%, 95% sequence identity to the nucleic acid sequence set forth in SEQ ID NO:5, wherein the nucleic acid comprises at least 20, 30, 40, 50, 100 nucleotides.

TIMP-4 has a molecular mass of 22 kDa and is 37% identical to TIMP-1 and 51% identical to TIMP-2 and -3 (Greene et al. 1996). TIMP-4 is the most neutral TIMP protein under physiological conditions (pH 7.4), having an isoelectric point of 7.34, compared with values of 8.00, 6.45 and 9.04 for human TIMP-1, TIMP-2 and TIMP-3, respectively (Wilde et al. 1994, Greene et al. 1996). The TIMP-4 gene is transcribed into 1.4 kb mRNA species (Olson et al. 1998). Of the calcified tissues, TIMP-4 has been detected in human cartilage (Huang et al. 2002).

Human TIMP-4 comprises the amino acid sequence set forth in SEQ ID NO:6 and is encoded by the nucleic acid sequence set forth in SEQ ID NO:7 (Accession No. NM_{—}003256). Thus, the inhibitor of the provided methods can comprise the amino acid sequence set forth in SEQ ID NO:6, or a biologically active fragment thereof. The inhibitor can also comprise an amino acid having at least 70%, 75%, 80%, 85%, 90%, 95% homology to the amino acid sequence set forth in SEQ ID NO:6, or a biologically active fragment thereof. The inhibitor of the provided methods can also comprise a nucleic acid encoding the amino acid sequence set forth in SEQ ID NO:6, or a biologically active fragment thereof. Thus, the inhibitor of the provided methods can comprise the nucleic acid sequence set forth in SEQ ID NO:7. The inhibitor can also comprise nucleic acid having at least 70%, 75%, 80%, 85%, 90%, 95% sequence identity to the nucleic acid sequence set forth in SEQ ID NO:7, wherein the nucleic acid comprises at least 20, 30, 40, 50, 100 nucleotides.

Each TIMP binds with both a different rate of interaction and affinity to a target MMP, usually in 1:1 or 2:2 stoichiometrical fashions. TIMP-1 inhibits MMP-1, MMP-3 and MMP-9 more effectively than TIMP-2 (Howard et al. 1991, Baragi et al. 1994, O'Connell et al. 1994, Nguyen et al. 1994). TIMP-2 inhibits proMMP-2 over 10-fold more effectively than TIMP-1 (Stetler-Stevenson et al. 1989a, Howard et al. 1991). However, TIMP-2 has a bi-functional effect on MMP-2 since MT1-MMP mediated proMMP-2 activation requires a tiny amount of TIMP-2 to make activation progress, whereas a greater concentration of TIMP-2 inhibits MMP-2 (Kinoshita et al. 1998). TIMP-3 inhibits at least MMP-2 and MMP-9 (Butler et al. 1999), whereas TIMP-4 is a good inhibitor for all classes of MMPs without remarkable preference for specific MMPs (Stratmann et al. 2001). TIMP-4 regulates MMP-2 activity both by inhibiting MT 1-MMP and by inhibiting activated MMP-2 (Bigg et al. 2001, Hernandez-Barrantes et al. 2001).

Other inhibitors of MMPs fall into three pharmacologic categories: 1) collagen peptidomimetics and nonpeptidomimetics, 2) tetracycline derivatives, and 3) bisphosphonates. A review of MMP inhibitor development can be found in Hidalgo M and Eckhardt, J Natl Cancer Inst. 93(3):178-93, which is hereby incorporated by reference in its entirety for this teaching.

Peptidomimetic MMP Inhibitors are pseudopeptide derivatives that have been synthesized to mimic the structure of collagen at the site where MMP binds to cleave it. The inhibitor binds reversibly at the active site of the MMP in a stereospecific manner and chelates the zinc atom on the enzyme activation site. Several zinc-binding groups have been tested for their ability to competitively inhibit MMP by binding at the active site; these groups include carboxylates, aminocarboxylates, sulfhydryls, derivatives of phosphoric acid, and hydroxamates. Most MMP inhibitors in clinical development are hydroxamate derivatives. Thus, the inhibitor of the provided methods can be a hydroxamate or hydroxamate derivative. Thus, “hydroxamate” is used herein to refer to both hydroxamates derivatives thereof. As a non limiting example, the hydroxamate can selected from the group consisting of BB-94, BB-1101, BB25-16, SE205, AG3340, and CGS 27023A.

Batimastat, the first MMP inhibitor evaluated in cancer patients, is a nonorally bioavailable low-molecular-weight hydroxamate. This compound is potent but relatively nonselective, with IC50 (concentration that causes 50% enzyme inhibition) values of less than 10 ng/mL for MMP-1,-2,-3,-7, and -9 inhibition. Marimastat is a synthetic low-molecular-weight MMP inhibitor that, in contrast to batimastat, is orally bioavailable, with an absolute bioavailability of 20%-50% in preclinical studies. The drug contains a collagen-mimicking hydroxamate structure that chelates the zinc ion at the active site of MMPs. Like batimastat, marimastat is relatively nonspecific, inhibiting the activity of MMP-1,-2,-3,-7, and -9 with IC50s of 2.5, 3, 115, 8, and 1.5 ng/mL, respectively.

Several nonpeptidic MMP inhibitors have been rationally synthesized on the basis of the three-dimensional x-ray crystallographic conformation of the MMP active site. Several of these molecules demonstrated antitumor activity in preclinical models and were selected for clinical development. The rational chemical design of MMP inhibitors made possible the synthesis of compounds with specific inhibitory activity against the MMP subtypes that predominate in certain diseases, such as cancer and arthritis. For example, AG3340, BAY 12-9566, and BMS-275291 were designed to be relatively selective inhibitors of MMP-2, whereas Ro 32-3555 was designed to be specific for MMP-1, which is frequently associated with osteoarticular diseases, and is thus being developed for arthritis. AG3340, BAY 12-9566, BMS-275291, and CGS 27023A are currently undergoing clinical evaluation in cancer patients.

BAY 12-9566 (Bayer) is an orally bioavailable biphenyl compound that is a potent inhibitor of MMP-2,-3, and -9, with an IC50 below 0.13 μg/mL. The compound was rapidly and substantially absorbed after oral administration, with an oral bioavailability of 70%-98%, and reached peak plasma concentrations at 0.5-2 hours after dosing, with evidence of enterohepatic recirculation. The pharmacokinetics of BAY 12-9566 in normal volunteers was linear at doses of up to 100 mg/day. Repeated administration of the drug resulted in increased clearance and thus a reduction in drug exposure.

AG3340 (Agouron Pharmaceuticals, Inc) is a nonpeptidic collagen-mimicking MMP inhibitor that was synthesized by use of a protein structure drug design program. The drug inhibits MMP-2,-9,-3, and -13, with IC50s of below 0.13 ng/mL. AG3340 is a low-molecular-weight compound that is lipophilic and crosses the blood-brain barrier. The agent has been administered on a continuous oral dosing schedule at doses that ranged from 2 to 100 mg/day given in two doses per day. Although treatment with AG3340 did not result in severe dose-limiting toxicity, doses above 25 mg/day induced musculoskeletal effects that required dose discontinuation in more than half of the subjects. At this dose, AG3340 can be safely combined with mitoxantrone/prednisone and carboplatin/paclitaxel.

BMS-275291 (Bristol-Myers Squibb Co) is an orally bioavailable MMP inhibitor in phase I clinical development. In preclinical studies, BMS-275291 demonstrated potent inhibitory activity against MMP-2 and MMP-9. This compound does not cleave the extracellular domain of the TNF receptor, which is thought to be responsible for some of the musculoskeletal effects of nonpeptidic MMP inhibitors.

CGS-27023A (Novartis Pharma AG) is a broad-spectrum inhibitor of MMPs. CGS-27023A has been evaluated in a phase I clinical trial administered orally on a continuous dosing schedule at doses ranging from 150 to 600 mg in divided doses. The major toxic effects, which were encountered at doses exceeding 300 mg twice a day, consisted of cutaneous and musculoskeletal toxicity. Pharmacokinetic analysis revealed that administration of CGS-27023 at clinically tolerable doses yielded plasma concentrations that were severalfold greater than the in vitro IC50s for MMP-2,-3, and -9 and that were sustained for longer than 10 hours after dosing.

Tetracycline derivatives inhibit not only the activity but also the production of MMPs and are thus being investigated for the treatment of disorders in which the MMP system becomes amplified, such as degenerative osteoarthritis, periodontitis, and cancer. This family of agents comprises both the classic tetracycline antibiotics, such as tetracycline, doxycycline, and minocycline, and as the newer tetracycline analogues that have been chemically modified to eliminate their antimicrobial activity (e.g., removal of the dimethylamino group from carbon-4 of the “A” ring). These agents inhibit the collagenases, MMP-1,-3, and -13, and the gelatinases, MMP-2 and -9, via multiple mechanisms, including 1) blocking the activity of mature MMPs by chelation of the zinc atom at the enzyme binding site, 2) interfering with the proteolitic activation of pro-MMP into their active form, 3) reducing the expression of MMPs, and 4) protecting MMPs from proteolytic and oxidative degradation. Some tetracycline derivatives have been evaluated in preclinical cancer models and have entered early clinical trials in patients with malignant diseases, including doxycycline and Col-3.

Bisphosphonates exert varied inhibitory effects on MMPs, including inhibition of their enzymatic activity. Clodronate, one of the most frequently used bisphosphonates, also inhibited the expression of the MT1-MMP protein and messenger RNA in the HT1080 fibrosarcoma cell line and decreased the invasion of C8161 melanoma and HT1080 fibrosarcoma cell lines through artificial basement membranes at IC50s ranging from 10 to 35 μg/mL (Teronen O, et al. Ann N Y Acad Sci 1999;878:453-65).

The inhibitor of the provided methods can be any MT1-MMP inhibitor that is known or provided herein, either alone, or in combination with any of the other MT1-MMP inhibitors. For example, the inhibitor of the provided methods can comprise a combination of TIMPs and hydroxamates. Thus, the inhibitor of the provided methods can comprise, for example, a combination of TIMP-2 and one or more of BB-94, BB-1101, BB25-16, SE205, AG3340, and CGS 27023A.

2. Sequence Similarities

It is understood that as discussed herein the use of the terms homology and identity mean the same thing as similarity. Thus, for example, if the word homology is used between, for example, two non-natural sequences it is understood that this is not necessarily indicating an evolutionary relationship between these two sequences, but rather is referring to the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define variants and derivatives of the disclosed genes and proteins is by defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

3. Compositions

The herein disclosed inhibitors of MT-MMP, such as MT1-MMP, can be combined with one or more substances that can be administered to the targets or subjects of the herein provided methods. For example, the disclosed inhibitors of MT-MMP, such as MT1-MMP, can be combined with one or more substances known in the art that can be administered to subjects with IDDM. The disclosed inhibitors of MT1-MMP can be combined with one or more substances that can be delivered to T cells. The substance can be a marker, therapeutic substance or targeting substance. Therapeutic substances include any compound, molecule, or composition of matter that will have a desired effect on the target tissue (e.g., pancreas, islets). Targeting substances include aptamers, antibodies, or fragment thereof. As an example, the targeting substance can target T cells. Thus, the targeting substance can target CD44.

Provided herein are compositions comprising an inhibitor of MT-MMP, such as MT1-MMP in a pharmaceutically acceptable carrier. The inhibitor can be any MMP inhibitor known or disclosed herein, alone or in combination. Thus, the inhibitor can be a native tissue inhibitor of MMP (TIMP). The TIMP can be TIMP-2. The TIMP can be TIMP-3. The TIMP can be TIMP-4. The inhibitor can also be a hydoxamate. The hydroxamate can be selected from the group consisting of BB-94, BB-1101, BB25-16, SE205, AG3340, and CGS 27023A. Thus, the inhibitor can be AG3340.

4. Pharmaceutical Carriers

The disclosed compositions can be administered in vivo in a pharmaceutically acceptable carrier. By “pharmaceutically acceptable” is meant a material that is not biologically or otherwise undesirable, i.e., the material can be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. For example, the carrier can be human albumin or human plasma.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

### B. METHODS

Provided herein are methods of inhibiting the transmigration of T cells through pancreatic capillary endothelium, comprising administering to the cells a composition comprising an inhibitor of membrane type matrix metalloproteinase (MT-MMP). The cells of the provided method can be in or from a subject in need of inhibition of transmigration of T cells through pancreatic capillary endothelium.

Matrix metalloproteinases (MMPs) are a family of enzymes that are responsible for the degradation of extracellular matrix components. Of the sixteen proteins reported to date, ten are normally found as soluble molecules. Several of the MMP proteins have been shown to be integral membrane proteins and have been named membrane type matrix metalloproteinase (MT-MMPs). The MT-MMP family is now known to contain at least three members, MT1-MMP, MT2-MMP and MT3-MMP also known as MMP14, MMP15 and MMP16 respectively. While each of these proteins contain a C-terminal transmembrane domain allowing localization to the cell surface they are independent in expression. These proteins are also unique from the other members of the MMP family in that they contain an 8 amino acid insert in the catalytic domain. While it appears that MT1-MMP is responsible for cleaving progelatinase A (MMP-2, 72 kda Type IV collagenase) to its active form, MT2-MMP and MT3-MMP can also play a role in the activation of proenzyme MMP-2. Thus, the MT-MMP inhibitor of the provided methods can be an inhibitor of MT1-MMP, MT2-MMP, MT3-MMP, or a combination thereof. Thus, the inhibitor can be an inhibitor of MT1-MMP. In one aspect, the inhibitor is specific for MT1-MMP. In another aspect, the inhibitor is specific for MT1-MMP, MT2-MMP and MT3-MMP. In another aspect, the inhibitor can inhibit MMPs non-specifically.

“Inhibit,” “inhibiting,” and “inhibition” mean to decrease an activity, response, condition, disease, or other biological parameter. This can include but is not limited to the complete ablation of the activity, response, condition, or disease. This can also include, for example, a 10% reduction in the activity, response, condition, or disease as compared to the normal, native or control level. Thus, the reduction can be, for example, a 10, 20, 30, 40, 50, 60, 70, 80, 90, 100% reduction, or any amount of reduction in between, as compared to native or control levels.

Also provided herein are methods of treating, inhibiting or preventing type I diabetes in a subject, comprising administering to the subject a composition comprising an inhibitor of MT-MMP.

In the context of a subject having a disease or condition, the terms “treating” or “treatment” are used to mean acting on the subject in an attempt to affect, alter, reduce, ameliorate, eliminate or abolish the disease or condition and/or some or all of the symptoms or effects of the disease or condition. For example, treatment can be a method of reducing the symptoms or effects of a disease or condition. Treatment can also be a method of reducing the disease or condition itself rather than just the symptoms or effects. The treatment can be, for example, any reduction from normal or native levels and can be but is not limited to the complete ablation of the disease, condition, or the symptoms of the disease or condition. For example, a disclosed method for treating type I diabetes is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100% reduction, or any amount of reduction in between, as compared to normal, native or control levels. As used throughout, “preventing” means to preclude, avert, obviate, forestall, stop, delay, or hinder something from happening, especially by advance planning or action.

Also provided are methods of identifying a molecule, comprising screening a candidate molecule for the ability to inhibit MT-MMP activity, and determining if the candidate molecule can inhibit the transmigration of T cells through pancreatic capillary endothelium.

Also provided are methods of identifying a molecule, comprising determining if a molecule that inhibits MT-MMP activity can inhibit the transmigration of T cells through pancreatic capillary endothelium.

Also provided are methods of immobilizing T cells on pancreatic capillary endothelium, comprising contacting the cells with a composition comprising an inhibitor of MT-MMP. The cells of the method can be in or from a subject identified as a subject in need of immobilization of T cells on pancreatic capillary endothelium.

Also provided are methods of treating a subject at risk of type I diabetes, comprising administering to the subject a composition comprising an inhibitor of MT-MMP.

As used herein, “subject” includes, but is not limited to, animals, plants, bacteria, viruses, parasites and any other organism or entity that has nucleic acid. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can to an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In the context of diabetes and the disclosed methods and compositions, it is understood that a subject is a subject that has or can have diabetes.

The subject of the herein provided methods can be diagnosed as having type I diabetes. The signs and symptoms of type 1 diabetes are related to the increased amounts of glucose in the blood, a condition referred to as hyperglycemia. The most common symptoms of type 1 diabetes include: increased urination, increased thirst, weight loss in spite of an increased appetite, fatigue and increased susceptibility to infections. Testing for diabetes involves drawing blood samples and measuring the glucose (sugar) levels within the blood. In a random glucose test, a sample of blood can be obtained and tested at any time. According to the American Diabetes Association (ADA), a random glucose level of greater than 200 mg/dl is indicative of diabetes when associated with typical symptoms of diabetes. In a fasting glucose test, a sample of blood is obtained following a period of not eating or drinking (except water) for at least eight hours. Blood is usually drawn early in the morning, before breakfast. According to the ADA, a fasting blood glucose level of 126 mg/dl or higher on two occasions is indicative of diabetes. The fasting blood glucose test is the most common test used for diagnosing diabetes. During an oral glucose tolerance test, a fasting blood sugar is obtained initially. The person is then asked to drink a sweet sugary beverage. Blood glucose levels are then obtained every 30 minutes for the next two hours. A blood glucose level below 140 mg/dl at two hours is considered normal. A blood glucose level of greater than 200 mg/dl at two hours is indicative of diabetes. A blood glucose level of 140 to 200 mg/dl at two hours indicates impaired glucose tolerance (or pre-diabetes). These individuals should be monitored and screened for diabetes in the future. Impaired glucose tolerance is also a risk factor for heart disease. Once the blood glucose level rises above 180 mg/dl, glucose begins to spill over into the urine. If there is sugar in the urine, a blood glucose test should be performed. Ketones are present in the urine when the body begins to break down an excessive amount of fat for energy. Ketones indicate that there is not enough insulin to prevent fat from leaving fat cells. The presence of ketones can indicate a serious and potentially lethal complication of type 1 diabetes.

The provided methods can result in an increase in T cell immobilization on the capillary endothelium surrounding pancreatic islet. CD44, via its interactions with endothelial hyaluronan, mediates T cell adhesion on the endothelium. CD44 is a target of MT1-MMP proteolysis in tumor cells, wherein MT1-MMP cleavage releases the extracellular domain of CD44 from cell surfaces and inactivates the CD44 cell receptor function. The provided methods inhibit MT1-MMP and thus promote CD44-mediated adhesion of T cells. Thus, in one aspect, the method can result in at least a 5%, 10%, 15%, 20%, 25%, 30%, 35%, 40% increase in T cell immobilization on the capillary endothelium surrounding pancreatic islet. In another aspect, the inhibitor of the provided methods can substantially immobilize the T cells on the islet endothelium.

The provided method can result in a reduction in T cell homing to the pancreas. In order to destroy pancreatic islets, insulin-specific CD8+ T cells (IS-CD8+ cells) must first home into the islets. For example, homing can be receptor-mediated. The provided methods inhibit MT1-MMP and promote CD44-mediated adhesion of T cells. This increased CD44 adhesion inhibits T cell homing into the pancreas. Thus, the method can result in at least a 5%, 10%, 15%, 20%, 25%, 30%, 35%, 40% reduction in T cell homing to the pancreas.

The terms “targeting” or “homing,” as used herein, can refer to the preferential movement, binding and/or accumulation of a targeted compound or composition, such as T cells or the disclosed compositions, at a site or a location as compared to a non-targeted compound or composition. For example, in the context of T cells, “homing” refers to the movement of T cells to a target tissue. In the context of in vivo administration to a subject, “targeting” or “homing” can refer to the preferential movement, binding, and/or accumulation of a compound or composition, such as the disclosed compositions, in or at, for example, target tissue, target cells, and/or target structures as compared to non-target tissue, cells and/or structures.

The term “target tissue” as used herein refers to an intended site for accumulation of a targeted compound or composition, such as T cells or the disclosed compositions, following administration to a subject. For example, the methods of the presently disclosed subject matter employ a target tissue comprising endometriosis.

As disclosed herein, the inhibitor of the provided methods can also promote regeneration of functional islets. The pathogenesis of IDDM involves the activation of autoimmune T cells followed by their homing into the pancreatic islets. In the islets, T cells directly destroy insulin-producing β cells. The provided methods inhibit T cell homing into the pancreas. In addition to inhibiting the destruction of islet cells, the provided methods allow the regeneration of functional islets. Thus, the method can result in at least a 5%, 10%, 15%, 20%, 25%, 30%, 35%, 40% regeneration of functional islets.

The T cell of the provided methods can be an insulin-specific, CD8-positive T cell (IS-CD8+ cell). In NOD/LtJ (NOD) mice, CD8+ cells are necessary for initiation of spontaneous diabetes, as NOD mice lacking expression of MHC class I are protected from the disease. Insulin-specific CD8+ T cells found within the infiltrates in the pancreata of prediabetic NOD mice recognize a peptide from insulin B chain amino acids 15-23 (SEQ ID NO:1) in the context of the MHC class I molecule.

1. Administration

The disclosed compositions can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. Thus, the disclosed compositions can be administered orally, parenterally (e.g., intravenously), by intramuscular injection, by intraperitoneal injection, transdermally, extracorporeally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type, such as T cells, via antibodies, receptors, or receptor ligands.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

i. Nucleic Acid Delivery

As disclosed herein, the provided methods can comprise the administration and uptake of exogenous DNA into the cells of a subject (i.e., gene transduction or transfection). For example, nucleic acids encoding the disclosed inhibitors can be delivered to cells. The disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (QIAGEN, Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding the desired MT-MMP inhibitor (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

2. Therapeutic Uses

Effective dosages and schedules for administering the compositions can be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of the provided compositions used alone might range from about 1 mg/kg to up to 100 mg/kg of body weight or more per day, including from about 1 mg/kg to about 10 mg/kg, depending on the factors mentioned above.

The pharmacological inhibition of MT1-MMP by the anti-cancer hydroxamate drugs including AG3340 will result in a favorable outcome for the IDDM patients. Because the inhibitors readily access cell surface-associated MT1-MMP in T cells in blood, the low concentrations of inhibitors are required in IDDM. In contrast, inhibition of MMPs in cancer required high concentrations of the inhibitors which have to be delivered to poorly angiogenic tumors. Further, the required dosages of the MMP inhibitors in IDDM will be below the levels which will cause side effects.

Following administration of a disclosed composition for treating, inhibiting, or preventing IDDM, the efficacy of the therapeutic composition can be assessed in various ways well known to the skilled practitioner. For instance, one of ordinary skill in the art will understand that a composition disclosed herein is efficacious in treating or inhibiting IDDM in a subject by observing a decrease in blood sugar.

### C. DEFINITIONS

It is understood that the disclosed methods and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an inhibitor” includes a plurality of such inhibitors, reference to “the inhibitor” is a reference to one or more inhibitor and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present methods and compositions, the particularly useful methods, devices, and materials are as described. Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the methods and compositions described herein. Such equivalents are intended to be encompassed by the following claims.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Inhibition of Membrane Type-1 Matrix Metalloproteinase by Cancer Drugs Interferes with the Homing of Diabetogenic T Cells into the Pancreas

Materials and Methods

Mice and Cells—NOD mice of NOD/LtJ strain were obtained from the Jackson Laboratory. IS-CD8⁺ T cells (insulin-specific, CD8-positive, Kd-restricted T cells of the TGNFC8 clone from the pancreas of NOD mouse) (Wong, F. S., et al. (2003)) were maintained in Click's medium supplemented with 5% fetal calf serum, 2×10⁻⁵ M β-mercaptoethanol, 20 mM penicillin-streptomycin, 3 mg/ml L-glutamine, and 5 units/ml recombinant murine interleukin-2 (Savinov, A. Y., et al. (2003)). Every 3 weeks IS-CD8⁺ cells were mixed with irradiated NOD splenocytes (2000 rads) loaded with the L¹⁵YLVCGERG²³ (SEQ ID NO:1) insulin B chain peptide (10 μg/ml) (Wong, F. S., et al. (1999)).

Induction of Diabetes in NOD Mice—IS-CD8⁺ cells were incubated both with and without AG3340 (50 μM or 21 μg/ml) for 2 h and then injected intravenously into the irradiated (725 rads, 24 h in advance), 5-8-week-old mice (1×10⁷ cells/animal). Mice were monitored for 21 days. On days 0, 2, 4, 6, 8, and 10 following the injection of the cells, mice received intraperitoneal injection with AG3340 (30 mg/kg or 1 mg/kg) or phosphate-buffered saline alone. The onset of diabetes was identified by assessing urine glucose levels with Diastix® strips (Bayer). Mice with urine glucose levels of >2000 mg/dl for 3 consecutive days were considered diabetic. AG3340 (molecular mass=421 Da) was obtained. The level of glucose in the urine closely follows that in the blood (Traisman, H. S., and Greenwood, R. D. (1973)). The measurement of glucose in urine is a widely accepted method to follow the development of diabetes in NOD mice (Pomerleau, D. P., et al. (2005)).

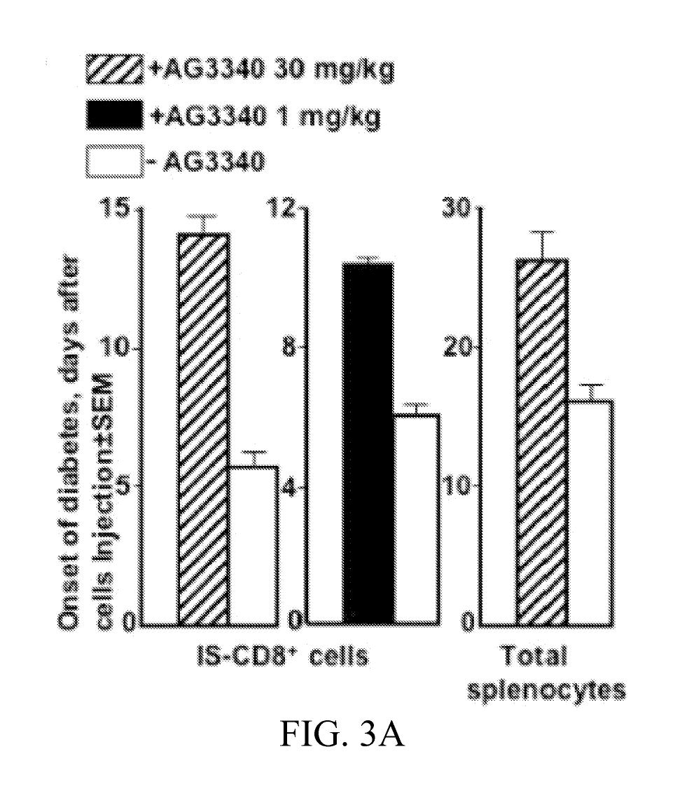

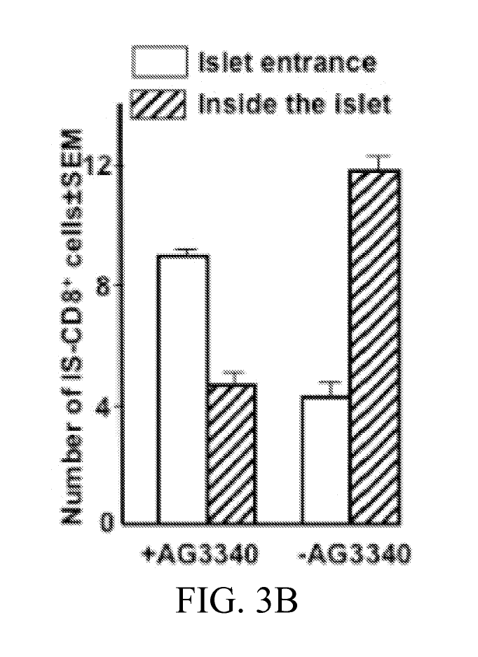

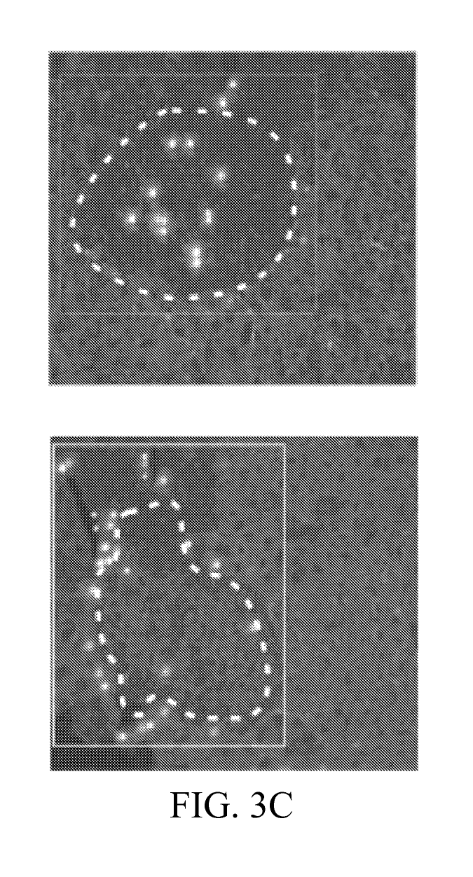

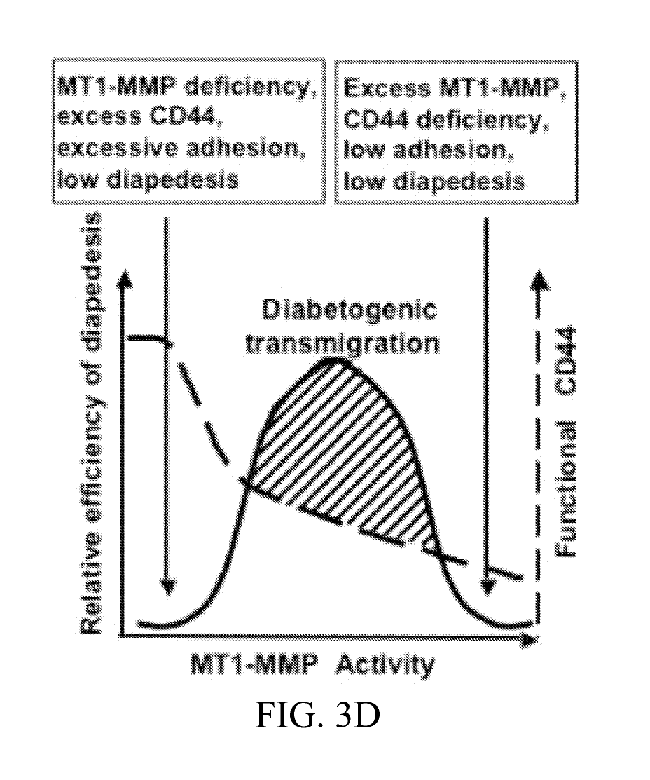

Fluorescent Tracing and Morphometric Analysis—For trafficking studies, IS-CD8⁺ T cells were incubated at 1×10⁷ cells/ml for 30 min at 37° C. in the dark in complete Click's medium containing 5% fetal calf serum and 0.0075 mg/ml of the fluorescent dye 1,1′-didodecyl-3,3,3′,3′-tetramethylindocarbocyanine perchlorate (DiI; Molecular Probes, Eugene, Oreg.). After incubation, the cells were washed three times with phosphatebuffered saline to remove excess DiI. Labeled IS-CD8⁺ cells (1×10⁷ cells) were injected intravenously in 0.2 ml of phosphate-buffered saline into irradiated (725 rads, 24 h in advance) NOD mice. Mice were sacrificed 24 h after injection of DiI-labeled cells. The spleen and the pancreata were excised and fixed in 0.1 M periodate-lysine-paraformaldehyde phosphate buffer. The organs were next sucrose-saturated, freeze-molded in OCT compound (Sakura Finetek Inc., Torrance, Calif.), and freeze-sectioned. 7-μm-thick cryostat sections of the entire pancreas were prepared at 60-μm intervals using a Leica CM1900 cryotom. Distribution of DiI-labeled CD8⁺ cells within the islets was examined using a fluorescent microscope. At least 100 individual islets per mouse (4-5 mice/experimental group) were examined. The characteristic morphology of the islets is easily identified on the cryosections. DiI-labeled cells were counted within the area relevant to each individual islet (see FIG. 3 for a representative image; the islet-relevant area is depicted by the solid white line, and the islet boundaries are shown by the white dotted line). The position of each IS-CD8⁺ labeled cell was determined relative to the islet boundary. The labeled cells localized within the islet boundary were considered to be “inside,” whereas the labeled cells adjacent to the islet but outside of the islet boundary were considered to be “at the entrance.” This method has been described in substantial detail (Savinov, A. Y., et al. (2003), and Savinov, A. Y., et al. (2001), which are herein incorporated by reference in their entirety for their teaching of these methods).

Treatment of Cells with MT1-MMP-CAT—The catalytic domain of MT1-MMP (MT1-MMP-CAT; 3 μg) was co-incubated for 2 h at 37° C. with IS-CD8⁺ cells (1×10⁷ cells) in 0.2 ml of 50 mM HEPES, 10 Mm CaCl₂, 0.5 mM MgCl₂, 50 μM ZnCl₂, and 0.01% Brij-35 buffer, pH 6.8. Where indicated, GM6001 (50 μM; Chemicon, Temecula, Calif.) was added to the samples. Following treatment, the cells were injected into the irradiated mice or used for DiI labeling, Western blotting, FACS analysis, and other analytical procedures (Savinov, A. Y., et al. (2003); Deryugina, E. I., et al. (2001); Rozanov, D. V., et al. (2001)).

Western Blotting—IS-CD8⁺ cells were surface biotinylated for 1 h at 4° C. with 0.1 mg/ml sulfo-NHS-LC-biotin (Pierce, Rockford, Ill.). Following washes to remove biotin, cells were co-incubated for 2 h with MT1-MMP-CAT in serum-free, unsupplemented Click's medium. The cells were then lysed with 50 mM N-octyl-β-D-glucopyranoside in phosphate-buffered saline supplemented with 1 mM CaCl₂, 1 mM MgCl₂, and protease inhibitor mixture containing phenylmethylsulfonyl fluoride (1 mM) and aprotinin, pepstatin, and leupeptin (1 μg/ml each). Biotin-labeled CD44 was captured from the cell lysate and from the medium aliquots on streptavidin-agarose beads. The captured samples were examined by Western blotting with the CD44 (clone IM7.8.1) antibody to determine the released, soluble, CD44 ectodomain in the medium samples and the residual, membrane-anchored, cellular CD44 in the cell lysates.

MT1-MMP-dependent MMP-2 Activation and Gelatin Zymography—IS-CD8⁺ cells (1×10⁶) were either allowed to adhere for 4 h in serum-free unsupplemented Click's medium to plastic coated with 2% gelatin or kept in solution. Under these experimental conditions, the vast majority of cells became attached to gelatin. In 4 h, media samples (30 μl each) were withdrawn and analyzed by gelatin zymography (Deryugina, E. I., et al. (2001)) to identify the proteolytic activity and the activation status of MMP-2 naturally synthesized by IS-CD8⁺ cells. Where indicated, cells were supplemented with external purified pro-MMP-2 (20 ng). Pro-MMP-2 was isolated from a conditioned medium of p2AHT2A72 cells derived from an HT1080 fibrosarcoma cell line sequentially transfected with the E1A and MMP-2 cDNAs (Strongin, A. Y., et al. (1995)).

Monoclonal Antibodies and FACS Analysis—IS-CD8⁺ cells were stained with the MT1-MMP (Ab815; Chemicon), CD44 (clone IM7.8.1), CD3 (clone 17A2), and CD49d (clone SG31) monoclonal antibodies (all from BD Biosciences, Rockville, Md.) and the CD29 monoclonal antibody (Chemicon), followed by staining with fluorescein isothiocyanate- or phycoerythrinconjugated secondary antibody (BD Biosciences), and then analyzed on a FACScan flow cytometer (Becton Dickinson, Franklin Lakes, N.J.). To determine the levels of CD44, IS-CD8⁺ cells were also stained with soluble fluorescently labeled hyaluronan (Sigma, St. Louis, Mo.). IS-C8⁺ cells were counterstained with phycoerythrin or fluorescein isothiocyanate-conjugated anti-CD8 antibody (Sigma).

Results

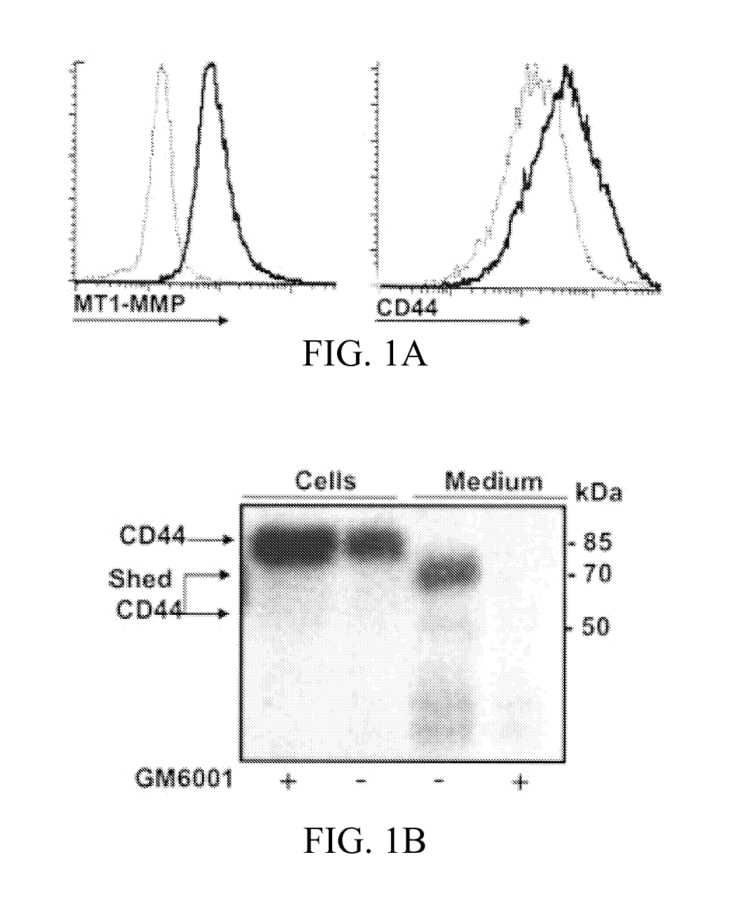

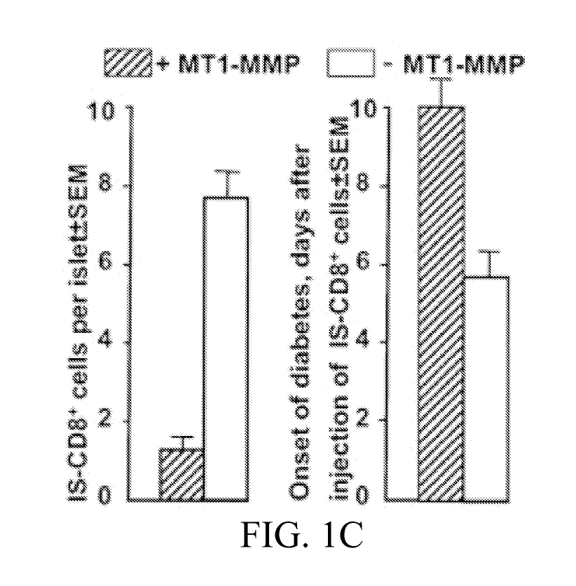

MT1-MMP Sheds Cellular CD44—It was determined that MT1-MMP proteolysis of T cell CD44 regulates adhesion and subsequent transmigration and homing of T cells into the pancreas. FACS analyses with MT1-MMP and CD44 antibodies and fluorescein isothiocyanate-labeled hyaluronan demonstrated the presence of high levels of cell surface-associated MT1-MMP and CD44 in IS-CD8⁺ cells in suspension (FIG. 1A). IS-CD8⁺ cells recognize an insulin B chain-derived L¹⁵YLVCGERG²³ (SEQ ID NO:1) peptide in the context of the Kd major histocompatibility complex class I molecule (Wong, F. S., et al. (1999)). Injection of IS-CD8⁺ cells induces diabetes in sub-lethally irradiated NOD/LtJ mice in 1 week (Savinov, A. Y., et al. (2003)). NOD mice are widely used as the best animal model of IDDM (Delovitch, T. L., and Singh, B. (1997)). Because the Ab815 antibody against MT1-MMP recognizes the hinge domain of the protease, FACS tests do not distinguish the pro-enzyme, the activated enzyme forms, and the inert complexes of MT1-MMP with tissue inhibitors of metalloproteinases including tissue inhibitor of metalloproteinase-2.

The levels of CD44 were significantly reduced in the majority of IS-CD8⁺ cells co-incubated with the external, purified, catalytically potent MT1-MMP-CAT (FIG. 1A). This treatment did not affect the levels of other T cell receptors including CD3, CD8, CD29, and CD49 or the viability of T cells. To support these observations, IS-CD8⁺ cells were also surface-labeled with membrane-impermeable biotin and then co-incubated with MT1-MMP-CAT. The liberated, soluble CD44 fragments were next captured on streptavidin-agarose beads and detected by Western blotting. These studies confirmed that during co-incubation of MT1-MMP-CAT with IS-CD8⁺ cells, the limited quantities of MT1-MMP-CAT released significant amounts of the digest fragments of cellular CD44 and reduced the residual levels of cell surface-associated CD44. These data correlate well with the results of the FACS analyses shown in FIG. 1A. Consistent with the role of MT1-MMP in the cleavage of CD44, GM6001 reversed the effect of MT1-MMP-CAT (FIG. 1B). IS-CD8⁺ cells co-incubated with MT1-MMP-CAT were also labeled with a fluorescent DiI dye and then injected into irradiated NOD mice. In 24 h, labeled cells were counted in the IDDM (Delovitch, T. L., and Singh, B. (1997)). Because the Ab815 antibody against MT1-MMP recognizes the hinge domain of the protease, FACS tests do not distinguish the pro-enzyme, the activated enzyme forms, and the inert complexes of MT1-MMP with tissue inhibitors of metalloproteinases including tissue inhibitor of metalloproteinase-2.

The levels of CD44 were significantly reduced in the majority of IS-CD8⁺ cells co-incubated with the external, purified, catalytically potent MT1-MMP-CAT (FIG. 1A). This treatment did not affect the levels of other T cell receptors including CD3, CD8, CD29, and CD49 or the viability of T cells. To support these observations, IS-CD8⁺ cells were also surface-labeled with membrane-impermeable biotin and then co-incubated with MT1-MMP-CAT. The liberated, soluble CD44 fragments were next captured on streptavidin-agarose beads and detected by Western blotting. These studies confirmed that during co-incubation of MT1-MMP-CAT with ISCD8⁺ cells, the limited quantities of MT1-MMP-CAT released significant amounts of the digest fragments of cellular CD44 and reduced the residual levels of cell surface-associated CD44. These data correlate well with the results of the FACS analyses shown in FIG. 1A. Consistent with the role of MT1-MMP in the cleavage of CD44, GM6001 (a potent hydroxamate inhibitor of MT1-MMP) reversed the effect of MT1-MMP-CAT (FIG. 1B).

IS-CD8⁺ cells co-incubated with MT1-MMP-CAT were also labeled with a fluorescent DiI dye and then injected into irradiated NOD mice. In 24 h, labeled cells were counted in the pancreatic islets. MT1-MMP proteolysis of CD44 caused a ˜4.5-fold decrease in cell homing and almost a 2-fold delay of the onset of diabetes in mice (FIG. 1C). These results indicated that cleavage of T cell CD44 by the external MT1-MMP-CAT decreased the number of the IS-CD8⁺ cells that were capable of adhering to hyaluronan of the pancreatic endothelium. Consequently, following co-incubation with MT1-MMP-CAT, the number of transmigrating cells was also low.

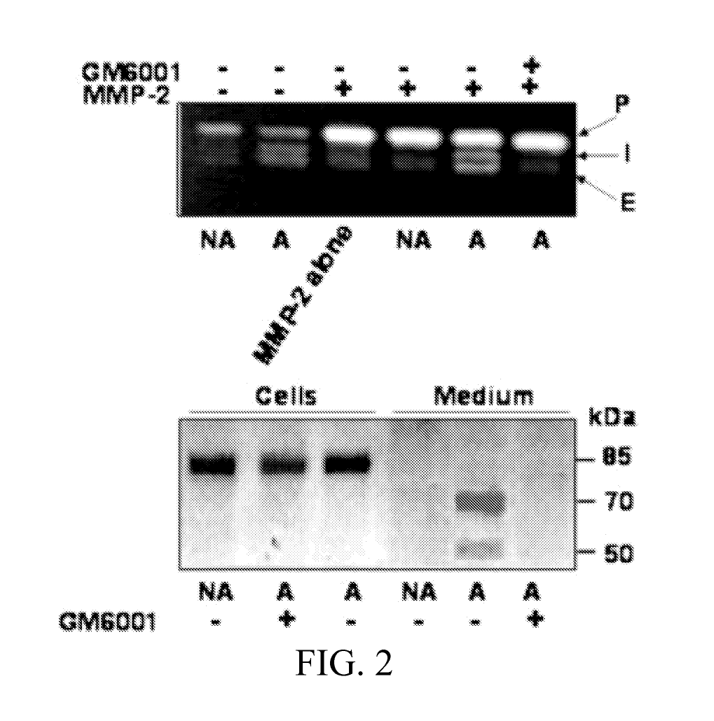

MT1-MMP Is Activated in Adherent IS-CD8⁺ Cells—Endogenous MT1-MMP is latent in non-adherent IS-CD8⁺ cells, whereas adhesion of IS-CD8⁺ cells induces the activation of MT1-MMP, the cleavage of CD44, and the stimulation of T cell transmigration. Thus, IS-CD8⁺ cells were capable of activating MMP-2, the enzyme known to be directly activated by MT1-MMP, only after their adhesion to gelatin (FIG. 2). Non-adherent cells did not activate MMP-2. In agreement, release of the CD44 proteolytic fragments into medium was detected only in adherent IS-CD8⁺ cells. CD44 remained intact in non-adherent cells. GM6001 blocked both the activation of MMP-2 and the shedding of CD44 in adherent cells (FIG. 2). These results indicate that MT1-MMP proteolysis of CD44 is induced only following adhesion of the diabetogenic cells to the substratum. Following adhesion, activated MT1-MMP could cleave CD44, and this event could promote the liberation of T cells, which is followed by the transmigration of T cells through the endothelium and their homing into the pancreatic islets. Conversely, inhibition of MT1-MMP could enhance the adhesion of T cells and repress their transmigration efficiency.

Inhibition of MT1-MMP Represses the Diabetogenicity of ISCD8⁺ Cells—To confirm the role of MT1-MMP in T cell transmigration, homing, and diabetogenesis, another potent hydroxamate inhibitor, AG3340 (Shalinsky, D. R., et al. (1999)) was used. AG3340 inhibits MT1-MMP with a Kᵢ in a sub-nanomolar range. AG3340 was used in cancer Phase I-III clinical trials (Hande, K. R., et al. (2004)). To evaluate AG3340, both IS-CD8⁺ cells and the splenocytes isolated from newly diabetic NOD mice (Savinov, A. Y., et al. (2003)) were used. The cells were co-incubated with AG3340 or left untreated and then injected in NOD mice, which then received AG3340 (30 mg/kg and 1 mg/kg) or solvent alone (control). AG3340 at a concentration as low as 1 mg/kg delayed the onset of diabetes ˜2-fold compared with the control (FIG. 3A).

In agreement, AG3340, by increasing the number of IS-CD8⁺ cells that remained firmly adhered to the hyaluronan of the pancreatic endothelium, significantly delayed the process of T cell entry into the pancreatic islets. IS-CD8⁺ cells co-incubated with AG3340 and labeled with DiI were injected into NOD mice. In 24 h, labeled IS-CD8⁺ cells were counted both at the periphery and inside the islets (FIG. 3, B and C). In the presence of AG3340, T cells were detected at the islet entrance, and a smaller number of cells were found inside the islets. In the absence of AG3340, the situation was reversed, and T cells efficiently transmigrated into the islets. These findings indicate that inhibition of T cell MT1-MMP is key to delaying the onset of diabetes in a well-accepted model of IDDM in rodents. The putative mechanism by which MT1-MMP proteolysis of CD44 regulates diapedesis is explained in FIG. 3D.

### 2. Example 2

### Targeting T Cell Membrane Proteinase in Spontaneous Type 1 Diabetes

Materials and Methods

Mice and cells—NOD mice of NOD/LtJ strain were obtained from the Jackson Laboratory. IS-CD8⁺ T cells (insulin-specific, CD8-positive, Kd-restricted T cells of the TGNFC8 clone from the pancreas of NOD mouse) (Wong, F. S., et al. (1996)) were maintained in Click's medium supplemented with 5% FCS, 2×10⁻⁵ M β-mercaptoethanol, 20 mM penicillin-streptomycin, 3 mg/ml L-glutamine, and 5 U/ml recombinant murine IL-2 (Savinov, A. Y., et al. (2003)). Every three weeks IS-CD8+ cells were mixed with irradiated NOD splenocytes (2000 Rad) loaded with the L¹⁵YLVCGERG²³ (SEQ ID NO:1) insulin B chain peptide (10 μg/ml) (Wong, F. S., et al. (1999)).

Newly diabetic NOD mice—NOD mice developed diabetes in approximately 5 months after the birth. The onset of spontaneous diabetes was identified by assessing urine glucose levels with Diastix® strips (Bayer). Mice with urine glucose levels >2000 mg/dl for three consecutive days were considered diabetic. The level of glucose in the urine follows closely to that in the blood (Traisman, H. S. & Greenwood, R. D. (1973). The measurement of the glucose in urine is a widely accepted method to follow the development of diabetes in NOD mice (Pomerleau, D. P., et al. (2005)). After development of diabetes, insulin (15-20 U/kg; one injection in every two-three days) was injected s.c. in mice. Control animals (6 mice/group) received insulin alone, while an experimental group (5 mice/group) received insulin s.c. jointly with AG3340 i.p. (1 mg/kg; one injection in every two-three days). AG3340 (molecular mass=421 D) was used. Injections were continued for 40 days and then mice were sacrificed. Leukocytes and granulated β cells were stained with H&E and aldehyde fuchsin, respectively, in the sections of paraformaldehyde-fixed, paraffin-embedded, pancreata. Islets (≧100/mouse) were scored as follows: 0, no lesions; 1, peri-insular leukocytic aggregates and, in addition, periductal infiltrates; 2, <25% islet destruction; 3, >25% islet destruction; and 4, totally destroyed islets. To identify hormone-producing cells the sections were stained with the antibody to insulin (Linco Research, St. Charles, Mo.) and glucagons (DacoCytomation, Carpinteria, Calif.) followed by species-specific secondary horseradish peroxidase-conjugated antibody and a 3,3′-diaminobenzidine substrate.

Fluorescent tracing and morphometric analysis—For trafficking studies, IS-CD8⁺ T cells were incubated at 1×10⁷ cells/ml for 30 min at 37° C. in the dark in the complete Click's medium containing 5% FCS and 0.0075 mg/ml of fluorescent dye 1,1′-didodecyl-3,3,3′,3′-tetramethylindocarbocyanine perchlorate (DiI; Molecular Probes). After incubation, the cells were washed three times with PBS to remove excess DiI. Labeled IS-CD8⁺ cells (1×10⁷ cells) were intravenously injected in 0.2 ml of PBS into irradiated (725 Rad, 24 h in advance) NOD mice. Mice were sacrificed 24 h after injection of DiI-labeled cells. Where indicated, the function-blocking antibody IM7.8.1 (BD Biosciences) against CD44 and AG3340 were each injected i.v in NOD mice (0.1 mg/animal and 1 mg/kg, respectively) 30 min before the i.v. injection of DiI-labeled IS-CD8⁺ T cells. In 24 h, the spleen and the pancreata were excised and fixed in 0.1 M periodate-lysine-paraformaldehyde phosphate buffer. The organs were next sucrose-saturated, freeze-molded in OCT compound (Sakura Finetek Inc.) and freeze-sectioned. 7-μm-thick cryostat sections of the entire pancreas were prepared at the 60 μm intervals using a Leica CM1900 cryotom. Distribution of DiI-labeled CD8⁺ cells within the islets was examined using a fluorescent microscope. At least 100 individual islets per mouse, 4-5 mice per each experimental group, were examined. The characteristic morphology of the islets is easily identified on the cryosections. DiI-labeled cells were counted within the area relevant to each individual islet. The position of each IS-CD8⁺ labeled cell was determined relative to the islet boundary. The labeled cells localized within the islet boundary were considered to be “inside”, while the labeled cells adjacent to the islet but outside of the islet boundary were considered to be “at the entrance”.

Western blotting—IS-CD8⁺ cells were surface biotinylated for 1 hr at 4° C. with 0.1 mg/ml of sulfo-NES-LC-biotin (Pierce). Following washes to remove biotin, the labeled cells were allowed to adhere in serum-free medium to plastic coated with 2% type I collagen/gelatin for 4 h or were kept in suspension. Under these experimental conditions, the vast majority of the cells became attached to gelatin. Where indicated, TIMP-1 and TIMP-2 (100 ng/ml each), and AG3340 (50 μM or 21 μg/ml) were added to the cells. The cells were then lysed with 50 mM N-octyl-β-D-glucopyranoside in PBS supplemented with 1 mM CaCl₂, 1 mM MgCl₂, and protease inhibitor cocktail containing phenylmethylsulfonyl fluoride (1 mM) and aprotinin, pepstatin, and leupeptin (1 μg/ml each). Biotin-labeled CD44 was captured from the cell lysate and from the medium aliquots on streptavidine-Agarose beads. The captured samples were examined by Western blotting with the CD44 (clone IM7.8.1) antibody to determine the released, soluble, CD44 ectodomain in the medium samples and the residual, membrane-anchored, cellular CD44 in the cell lysates.

MT1-MMP-dependent MMP-2 activation and gelatin zymography—IS-CD8⁺ cells (1×10⁶) were either allowed to adhere for 4 h in serum-free unsupplemented Click's medium to the plastic coated with 2% gelatin or kept in solution. In 18 h, media samples (30 μl each) were withdrawn and analyzed by gelatin zymography (Deryugina, E. I., et al. (2001)) to identify the proteolytic activity and the activation status of MMP-2 naturally synthesized by IS-CD8⁺ cells. Where indicated, the cells were supplemented with external purified pro-MMP-2 (20 ng), TIMP-1 and TIMP-2 (100 ng/ml each), and AG3340 (50 μM or 21 μg/ml). Pro-MMP-2 was isolated from a conditioned medium of p2AHT2A72 cells derived from an HT1080 fibrosarcoma cell line sequentially transfected with the E1A and MMP-2 cDNAs (Strongin, A. Y., et al. (1995)).

Results and Discussion

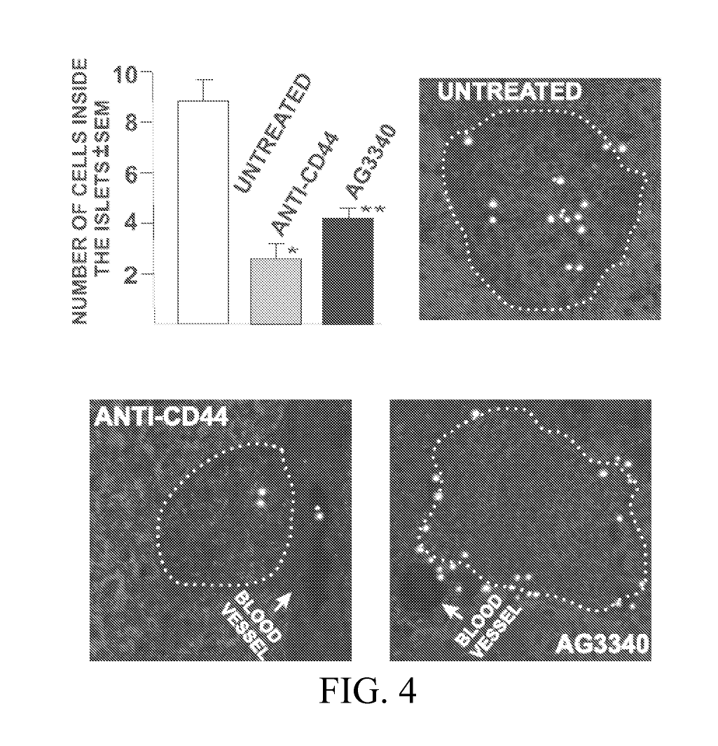

CD44 is a major adhesion receptor in diabetogenic T cells—To quantitatively assess the role of CD44 in the homing of T cells, NOD mice were irradiated at 725 Rad. In 24 h, a function-blocking antibody against CD44 and AG3340 were each injected in mice. After 30 min, this injection was followed by the i.v. injection of IS-CD8+ T cells labeled with a fluorescent dye DiI. Mice were sacrificed 24 h following injection of the cells. The pancreata were excised and freeze-sectioned. Distribution of DiI-labeled IS-CD8⁺ cells within the islets was examined using a fluorescent microscope. DiI-labeled cells were counted within the area relevant to each individual islet. FIG. 4 shows that IS-CD8⁺ cells efficiently homed inside the islets of the control mice, while the blocking of the CD44 functionality drastically reduced the efficiency of T cell homing. It was thus confirmed that CD44-mediated adhesion (Weiss, L., et al. (2000)) plays a highly significant role in the homing of T cells to the islets. AG3340 (by inhibiting the MT1-MMP-mediated proteolysis of CD44 in the adherent T cells) also caused a 50% reduction in the T cell homing. Representative images show the main difference between anti-CD44 and AG3340: the first suppressed the adhesion of T cells and, therefore, diminished the homing of DiI-labeled cells, while the second incapacitated the adherent T cells on the pancreatic endothelium at the islet's entrance.

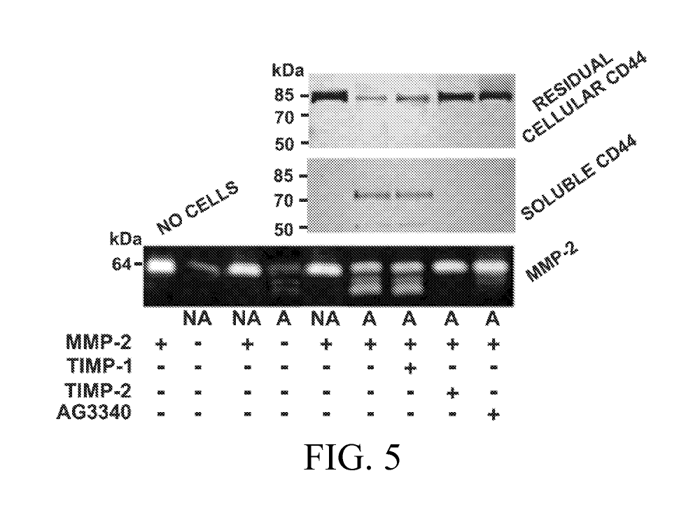

A causal link between MT1-MMP and CD44 shedding—It was next determined the significance of MT1-MMP activity in the shedding of CD44 and a causal link was identified between the two in the adherent IS-CD8⁺ cells. For these purposes, IS-CD8⁺ cells were surface biotinylated with membrane-impermeable biotin and the labeled cells were then either allowed to adhere to a gelatin-coated plastic or kept in solution. The cells were then lysed and biotin-labeled CD44 was captured from the cell lysate and from the medium aliquots on streptavidine-agarose beads. The captured samples were examined by Western blotting to measure both the quantities of the released, soluble, CD44 ectodomain in the medium samples and the residual, membrane-anchored, cellular CD44 in the cell lysates. In addition, media samples were analyzed by gelatin zymography to identify the activation status of MMP-2 which is naturally synthesized by IS-CD8⁺ cells. MMP-2 is an enzyme that is known to be directly activated by MT1-MMP (Egeblad, M. & Werb, Z. (2002); Strongin, A. Y., et al. (1995)). Where indicated, cells were supplemented with external, purified pro-MMP-2. TIMP-2 (a potent inhibitor of MT1-MMP), TIMP-1 (a poor inhibitor of MT1-MMP) and AG3340 were each added to the cell samples to distinguish the role of MT1-MMP from the putative effect imposed by the other individual cell surface-associated proteases (Will, H., et al. (1996)) (FIG. 5).

Consistent with observations in Example 1, endogenous MT1-MMP was latent in non-adherent cells, while adhesion of T cells induces the activation of MT1-MMP, the subsequent activation of MMP-2, and the cleavage of CD44. Thus, IS-CD8⁺ cells, only after their adhesion to gelatin, were capable of activating MMP-2. Non-adherent cells did not activate MMP-2. In agreement, the release of the CD44 fragments into the medium was detected only in adherent IS-CD8⁺ cells. CD44 remained intact in non-adherent cells. AG3340 and TIMP-2 each fully blocked both the activation of MMP-2 and the shedding of CD44 in adherent cells. In contrast, TIMP-1 had no effect on MMP-2 activation. TIMP-1 demonstrated a minor but noticeable inhibition of CD44 proteolysis. These results confirmed that MT1-MMP is the main, rather than the only, individual mediator of CD44 shedding in T cells. Other proteases (e.g., ADAMs) (Nakamura, H., et al. (2004)) are also involved in the proteolysis of CD44. The combined effect of these other proteases, however, is insignificant when compared to that of MT1-MMP (FIG. 5).

Inhibition of MT1-MMP proteolysis of CD44 in spontaneous IDDM—To assess if AG3340 has a potential for therapeutic translation, this inhibitor was used in female NOD mice with developed spontaneous diabetes. Control animals received insulin alone, while the experimental group received insulin jointly with AG3340. The dose of AG3340 that was used was an order of magnitude lower compared to the lowest dosage used in the phase I trials in cancer (Hande, K. R., et al. (2004)). In 40 days, the pancreata were excised, sectioned and the islets were analyzed and graded according to the observed insulitis. The sections were also stained with antibodies to insulin and glucagon to identify the functionality of the surviving and the newly formed islets (Luo, X., et al. (2005)).

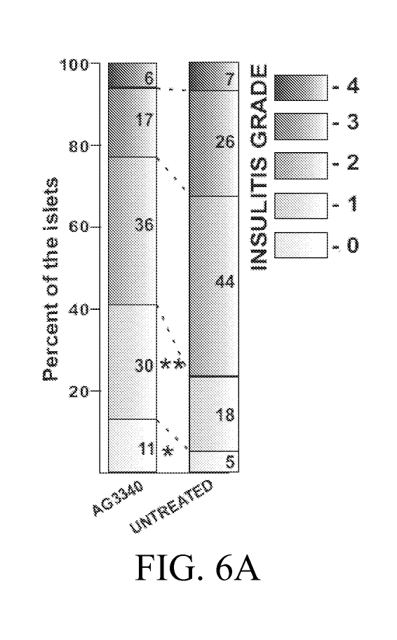

A short-term treatment with AG3340 was not sufficient to return overtly diabetic NOD mice to normoglycemia. There was, however, a significant and promising reduction in the severity of insulitis compared to the control (FIG. 6). AG3340 caused an increase in the number of the intact islets and the islets with limited peri-islet insulitis. Excitingly, AG3340 caused a de novo formation of the islet-like structures in the pancreatic parenchyma. These small, regenerating, islets were free from mononuclear infiltration and produced insulin (FIG. 6) and glucagon, thus, providing evidence of the functional regeneration of the hormone-secreting α and β cells. In contrast, there was an intensive mononuclear infiltration, evident destruction of the islets and a strongly reduced hormone production in the untreated control. These findings are consistent with the role of AG3340 in the control of the CD44-MT1-MMP axis in adoptively transferred T cell diapedesis. Overall, these results indicate that AG3340 caused diabetes protection by effectively controlling an islet-destructive autoimmunity and stimulating the functional regeneration of the insulin-producing β cells and the pancreatic islets.

### 3. Example 3

### Defining the Roles of T Cell Membrane Proteinase and CD44 in Type 1 Diabetes

The specific role of T cell MT1-MMP in IDDM: MMP-2, MMP-12 and MT1-MMP were up-regulated in diabetic male and high-fat-fed female Zucker diabetic fatty rats as compared to their non-diabetic lean counterparts (Zhou, Y. P., et al. 2005). PD166793 [(S)-2-(4′-bromo-biphenyl-4-sulfonylamino)-3-methyl-butyric acid] (a broad-range inhibitor with EC50 values of 6100 nM, 47 nM, 12 nM, 7200 nM, 7900 nM, 8 nM and 240 nM against MMP-1, MMP-2, MMP-3, MMP-7, MMP-9, MMP-13 and MT1-MMP, respectively) O'Brien, P. M., et al. 2000; Peterson, J. T., et al. 2001) preserved β cell mass, presumably, by decreasing the turnover of islet extracellular matrix molecules.

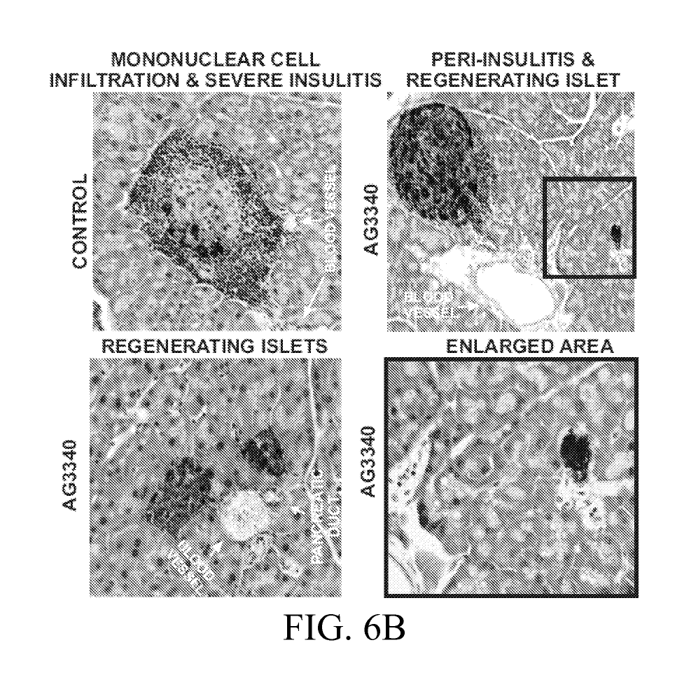

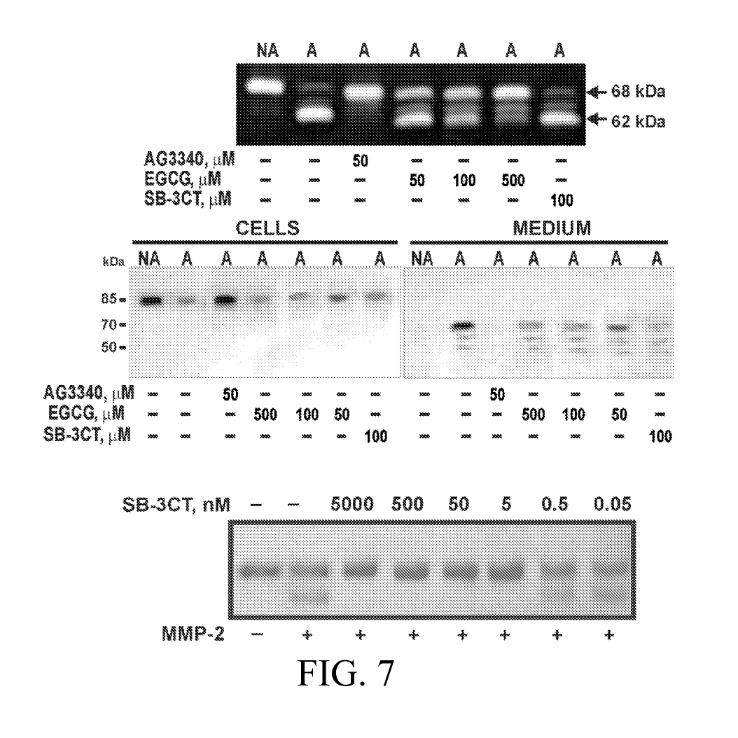

To validate the specific role of T cell MT1-MMP as well as to elucidate the potential significance of other MMPs in the NOD model of IDDM, AG3340 and two additional inhibitors, EGCG and SB-3CT were used. While both EGCG and SB-3CT are poor inhibitors of MT1-MMP, they are capable of targeting MMPs distinct from MT1-MMP. To determine if these two inhibitors were potent in inhibiting CD44 shedding, IS-CD8+ T cells were surface biotinylated with membrane-impermeable sulfo-NHS-LC-biotin. The labeled cells were then allowed either to adhere to a gelatin-coated plastic or were kept in solution. The cells were then lysed and biotin-labeled CD44 was captured from the cell lysate and from the medium aliquots on streptavidine-Agarose beads. The captured samples were examined by Western blotting to measure both the quantities of the released, soluble, CD44 ectodomain in the medium samples and the residual, membrane-anchored, cellular CD44 in the cell lysates. In addition, media samples were analyzed by gelatin zymography to identify the activation status of MMP-2. MMP-2 is an enzyme that is known to be directly activated by MT1-MMP (Strongin, A. Y., et al. 1995). Where indicated, cells were supplemented with AG3340, SB-3CT and EGCG (FIG. 7). Endogenous MT1-MMP was latent in non-adherent cells, while the adhesion of T cells induced the activation of MT1-MMP, the subsequent activation of MMP-2, and the cleavage of CD44. Non-adherent cells did not activate MMP-2 and they are incapable of efficient CD44 shedding. AG3340 fully blocked both the activation of MMP-2 and the shedding of CD44 in adherent cells. In contrast, SB-3CT (a poor inhibitor of MT1-MMP) had no effect on either MMP-2 activation or CD44 shedding while only an exceedingly high, 500 mM, concentration of EGCG demonstrated a partial inhibition of MMP-2 activation without any significant effect on CD44 proteolysis.

In contrast, SB-3CT was highly potent in inhibiting the MMP-2 proteolysis of α1-antitrypsin (a sensitive and readily available protein substrate of MMPs) (Li, W., et al. 2004; Mast, A. E., et al. 1991) and converting this 61 kDa serpin into a 55 kDa degradation fragment that represents the N-terminal portion of the α1-antitrypsin molecule. Thus, nanomolar range concentrations of SB-3CT totally blocked the cleavage of α1-antitrypsin in vitro (FIG. 7).

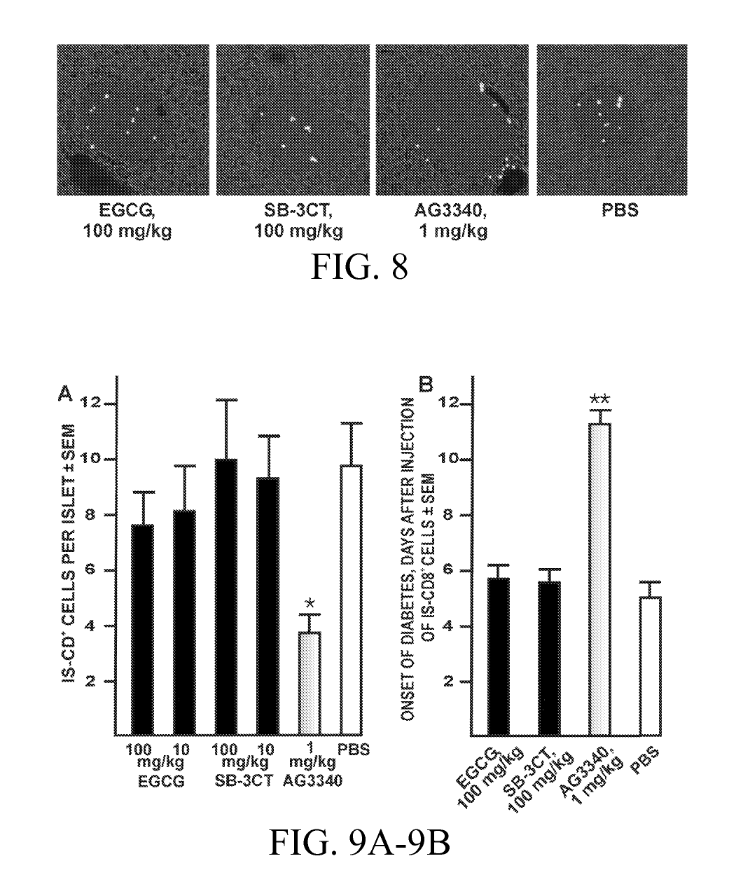

To determine the anti-diabetic potential of SB-3CT and EGCG relative to that of AG3340, NOD mice received an i.p. injection of the indicated concentrations of inhibitors. IS-CD8+ cells pre-labeled with a fluorescence dye, didodecyl-tetramethylindocarbocyanine perchlorate (DiI) and then were injected i.v. in NOD mice. In 24 h, labeled IS-CD8+ cells were counted both at the periphery and inside the islets (FIG. 8). In the absence of AG3340 T cells efficiently transmigrated into the islets. In the presence of AG3340 the situation was reversed and many more T cells were detected at the islet entrance and several-fold less cells were found inside the islets. In contrast, SB-3CT and EGCG, both of which were used at much higher concentrations than AG3340, did not affect the homing of IS-CD8+ cells.

To further corroborate these results, IS-CD8+ cells were injected in NOD mice. 30 min prior to cell injection, mice received either the inhibitors or PBS (control) i.p. The inhibitor injections continued every other day until mice developed diabetes. AG3340 at a concentration as low as 1 mg/kg delayed the onset of diabetes approximately 2-fold compared to the control (FIG. 9). In contrast, there was no delay of the transferred diabetes onset in mice which received SB-3CT and EGCG, both of which are potent inhibitors of MMPs other than MT1-MMP.

In a transfer diabetes model in NOD mice only AG3340, the antagonist of MT1-MMP, delivered clinically-relevant effects. Because of the wide-range specificity of the MMP inhibitors, only a simultaneous assessment of AG3340, SB-3CT and EGCG permitted the conclusion that T cell MT1-MMP plays a significant role in IDDM while the combined effect of all other MMPs, including MMP-2 and MMP-9, both of which are efficiently inhibited by SB-3CT, is far less important. These results confirm the functional importance of the MT1-MMP-CD44 axis in mediating the efficiency of transendothelial migration and the homing of diabetogenic T cells to the pancreatic islets.

Potential clinical relevance of targeting MT1-MMP in IDDM: Low dosages of AG3340 injected jointly with insulin reduced the diabetogenic efficiency of T cells, immobilized T cells on the endothelium, repressed the homing of diabetogenic T cells into the pancreatic islets, and reduced insulitis and mononuclear cell infiltration in acutely diabetic NOD mice. These combined events promoted the recovery of the insulin-producing β cells in diabetic NOD mice with freshly developed IDDM. Endothelial precursor stem cells rather than the β-cells themselves are the source of the regenerated, functional, β-cells.

To prove that insulin-producing β-cells were regenerated, NOD mice were allowed to develop IDDM. Diseased mice then received insulin alone or insulin jointly with AG3340 for 40 days. Insulin injections were then suspended. Mice which after the onset of the disease received insulin became hyperglycemic in a matter of 2-3 days and were then sacrificed according to NIH guidelines. In contrast, mice which received insulin jointly with the inhibitor restored the pool of insulin-producing β-cells. When insulin injection were cancelled, this β-cell pool was sufficient for the survival of these mice which continued to be normoglycemic/mildly hyperglycemic for several weeks without the use of external insulin.

### E. REFERENCES

Annabi, B., Lachambre, M. P., Bousquet-Gagnon, N., Page, M., Gingras, D. and Beliveau, R. (2002) Green tea polyphenol (−)-epigallocatechin 3-gallate inhibits MMP-2 secretion and MT1-MMP-driven migration in glioblastoma cells. Biochim. Biophys. Acta 1542, 209-220.
Apte S S, Mattei M G & Olsen B R (1994) Cloning of the cDNA encoding human tissue inhibitor of metalloproteinases-3 (TIMP-3) and mapping of the TIMP3 gene to chromosome 22. Genomics 19: 86-90.
Apte S S, Olsen B R & Murphy G (1995) The gene structure of tissue inhibitor of metalloproteinases (TIMP)-3 and its inhibitory activities define the distinct TIMP gene family. J Biol Chem 270: 14313-8.
Baragi V M, Fliszar C J, Conroy M C, Ye Q Z, Shipley J M & Welgus H G (1994) Contribution of the C-terminal domain of metalloproteinases to binding by tissue inhibitor of metalloproteinases. C-terminal truncated stromelysin and matrilysin exhibit equally compromised binding affinities as compared to full-length stromelysin. J Biol Chem 269: 12692-7.
Bigg H F, Morrison C J, Butler G S, Bogoyevitch M A, Wang Z, Soloway P D & Overall C M (2001) Tissue inhibitor of metalloproteinases-4 inhibits but does not support the activation of gelatinase A via efficient inhibition of membrane type 1-matrix metalloproteinase. Cancer Res 61: 3610-8.
Bonfil, R. D., Sabbota, A., Nabha, S., Bernardo, M. M., Dong, Z., Meng, H., Yamamoto, H., Chinni, S. R., Lim, I. T., Chang, M., Filetti, L. C., Mobashery, S., Cher, M. L. and Fridman, R. (2006) Inhibition of human prostate cancer growth, osteolysis and angiogenesis in a bone metastasis model by a novel mechanism-based selective gelatinase inhibitor. Int. J. Cancer 118, 2721-2726.
Boone T C, Johnson M J, De Clerck Y A & Langley K E (1990) cDNA cloning and expression of a metalloproteinase inhibitor related to tissue inhibitor of metalloproteinases. Proc Natl Acad Sci USA 87: 2800-4.
Butcher, E. C., Williams, M., Youngman, K., Rott, L. and Briskin, M. (1999) Lymphocyte trafficking and regional immunity. Adv. Immunol. 72, 209-253.
Butler G S, Apte S S, Willenbrock F & Murphy G (1999) Human tissue inhibitor of metalloproteinases 3 interacts with both the N— and C-terminal domains of gelatinases A and B. Regulation by polyanions. J Biol Chem 274: 10846-51.
Cappuzzo, F., Bartolini, S. and Crino, L. (2003) Emerging drugs for non-small cell lung cancer. Expert Opin. Emerg. Drugs 8, 179-192.
Carmichael D F, Sommer A, Thompson R C, Anderson D C, Smith C G, Welgus H G & Stricklin G P (1986) Primary structure and cDNA cloning of human fibroblast collagenase inhibitor. Proc Natl Acad Sci USA 83: 2407-11.
Chiang, W. C., Wong, Y. K., Lin, S. C., Chang, K. W. and Liu, C. J. (2006) Increase of MMP-13 expression in multi-stage oral carcinogenesis and epigallocatechin-3-gallate suppress MMP-13 expression. Oral Dis. 12, 27-33.
Chong, A. S., Shen, J., Tao, J., Yin, D., Kuznetsov, A., Hara, M. and Philipson, L. H. (2006) Reversal of diabetes in non-obese diabetic mice without spleen cell-derived beta cell regeneration. Science 311, 1774-1775.
Cichy, J. and Pure, E. (2003) The liberation of CD44. J. Cell Biol. 161, 839-843.
Clark I M, Rowan A D, Edwards D R, Bech-Hansen T, Mann D A, Bahr M J & Cawston T E (1997) Transcriptional activity of the human tissue inhibitor of metalloproteinases 1 (TIMP-1) gene in fibroblasts involves elements in the promoter, exon 1 and intron 1. Biochem J 324: 611-7.
De Clerck Y A, Darville M I, Eeckhout Y & Rousseau G G (1994) Characterization of the promoter of the gene encoding human tissue inhibitor of metalloproteinases-2 (TIMP-2). Gene 139: 185-91.
Dean G, Young D A, Edwards D R & Clark I M (2000) The human tissue inhibitor of metalloproteinases (TIMP)-1 gene contains repressive elements within the promoter and intron 1. J Biol Chem 275: 32664-71.
DeGrendele, H. C., Estess, P. & Siegelman, M. H. (1997) Science 278, 672-675.
Delovitch, T. L., and Singh, B. (1997) Immunity 7, 727-738.
Deryugina, E. I., Ratnikov, B., Monosov, E., Postnova, T. I., DiScipio, R., Smith, J. W. & Strongin, A. Y. (2001) Exp. Cell Res. 263, 209-223.
Egeblad, M. & Werb, Z. (2002) Nat. Rev. Cancer 2, 161-174.
El Bedoui, J., Oak, M. H., Anglard, P. and Schini-Kerth, V. B. (2005) Catechins prevent vascular smooth muscle cell invasion by inhibiting MT1-MMP activity and MMP-2 expression. Cardiovasc. Res. 67, 317-325.
Estess, P., DeGrendele, H. C., Pascual, V. and Siegelman, M. H. (1998) Functional activation of lymphocyte CD44 in peripheral blood is a marker of autoimmune disease activity. J. Clin. Invest. 102, 1173-1182.
Fernandez-Catalan C, Bode W, Huber R, Turk D, Calvete J J, Lichte A, Tschesche H & Maskos K (1998) Crystal structure of the complex formed by the membrane type 1-matrix metalloproteinase with the tissue inhibitor of metalloproteinases-2, the soluble progelatinase A receptor. Embo J 17: 5238-48.
Gallegos, A. M. and Bevan, M. J. (2004) Driven to autoimmunity: the nod mouse. Cell 117, 149-151.
Gomis-Ruth F X, Maskos K, Betz M, Bergner A, Huber R, Suzuki K, Yoshida N, Nagase H, Brew K, Bourenkov G P, Bartunik H & Bode W (1997) Mechanism of inhibition of the human matrix metalloproteinase stromelysin-1 by TIMP-1. Nature 389: 77-81.
Greene J, Wang M, Liu Y E, Raymond L A, Rosen C & Shi Y E (1996) Molecular cloning and characterization of human tissue inhibitor of metalloproteinase 4. J Biol Chem 271: 30375-80.
Gu, Z., Cui, J., Brown, S., Fridman, R., Mobashery, S., Strongin, A. Y. and Lipton, S. A. (2005) A highly specific inhibitor of matrix metalloproteinase-9 rescues laminin from proteolysis and neurons from apoptosis in transient focal cerebral ischemia. J. Neurosci. 25, 6401-6408.
Hammani K, Blakis A, Morsette D, Bowcock A M, Schmutte C, Henriet P & DeClerck Y A (1996) Structure and characterization of the human tissue inhibitor of metalloproteinases-2 gene. J Biol Chem 271: 25498-505.
Hande, K. R., Collier, M., Paradiso, L., Stuart-Smith, J., Dixon, M., Clendeninm, N., Yeun, G., Alberti, D., Binger, K. & Wilding, G. (2004) Clin. Cancer Res. 10, 909-915.
Hao, E., Tyrberg, B., Itkin-Ansari, P., Lakey, J. R., Geron, I., Monosov, E. Z., Barcova, M., Mercola, M. and Levine, F. (2006) Beta-cell differentiation from nonendocrine epithelial cells of the adult human pancreas. Nat. Med. 12, 310-316.
Hernandez-Barrantes S, Shimura Y, Soloway P D, Sang Q A & Fridman R (2001) Differential roles of TIMP-4 and TIMP-2 in pro-MMP-2 activation by MT1-MMP. Biochem Biophys Res Commun 281: 126-30.
Homann, D. & von Herrath, M. (2004) Clin. Immunol. 112, 202-209.
Howard E W, Bullen E C & Banda M J (1991) Preferential inhibition of 72- and 92-kDa gelatinases by tissue inhibitor of metalloproteinases-2. J Biol Chem 266: 13070-5.
Huang W, Li W Q, Dehnade F & Zafarullah M (2002) Tissue inhibitor of metalloproteinases-4 (TIMP-4) gene expression is increased in human osteoarthritic femoral head cartilage. J Cell Biochem 85: 295-303.
Ikejiri, M., Bernardo, M. M., Meroueh, S. O., Brown, S., Chang, M., Fridman, R. and Mobashery, S. (2005) Design, synthesis, and evaluation of a mechanism-based inhibitor for gelatinase A. J. Org. Chem. 70, 5709-5712.
Ishiguro K, Yamashita K, Nakagaki H, Iwata K & Hayakawa T (1994) Identification of tissue inhibitor of metalloproteinases-1 (TIMP-1) in human teeth and its distribution in cementum and dentin. Arch Oral Biol 39: 345-9.
Itoh, Y. and Seiki, M. (2006) MT1-MMP: a potent modifier of pericellular microenvironment. J. Cell. Physiol. 206, 1-8.
Jin, L., Hope, K. J., Zhai, Q., Smadja-Joffe, F. and Dick, J. E. (2006) Targeting of CD44 eradicates human acute myeloid leukemic stem cells. Nat. Med. 12, 1167-1174.
Kajita, M., Itoh, Y., Chiba, T., Mori, H., Okada, A., Kinoh, H. and Seiki, M. (2001) Membrane-type 1 matrix metalloproteinase cleaves CD44 and promotes cell migration. J. Cell Biol. 153, 893-904.
Kinoshita T, Sato H, Okada A, Ohuchi E, Imai K, Okada Y & Seiki M (1998) TIMP-2 promotes activation of progelatinase A by membrane-type 1 matrix metalloproteinase immobilized on agarose beads. J Biol Chem 273: 16098-103.
Krause, D. S., Lazarides, K., von Andrian, U. H. and Van Etten, R. A. (2006) Requirement for CD44 in homing and engraftment of BCR-ABL-expressing leukemic stem cells. Nat. Med. 12, 1175-1180.
Langton K P, Barker M D & McKie N (1998) Localization of the functional domains of human tissue inhibitor of metalloproteinases-3 and the effects of a Sorsby's fundus dystrophy mutation. J Biol Chem 273: 16778-81.
Li, W., Savinov, A. Y., Rozanov, D. V., Golubkov, V. S., Hedayat, H., Postnova, T. I., Golubkova, N. V., Linli, Y., Krajewski, S. and Strongin, A. Y. (2004) Matrix metalloproteinase-26 is associated with estrogen-dependent malignancies and targets alpha1-antitrypsin serpin. Cancer Res. 64, 8657-8665.
Luo, X., Yang, H., Kim, I. S., Saint-Hilaire, F., Thomas, D. A., De, B. P., Ozkaynak, E., Muthukumar, T., Hancock, W. W., Crystal, R. G. & Suthanthiran, M. (2005) Transplantation 79, 1091-1096.
Mast, A. E., Enghild, J. J., Nagase, H., Suzuki, K., Pizzo, S. V. and Salvesen, G. (1991) Kinetics and physiologic relevance of the inactivation of alpha 1-proteinase inhibitor, alpha 1-antichymotrypsin, and antithrombin III by matrix metalloproteinases-1 (tissue collagenase), -2 (72-kDa gelatinase/type IV collagenase), and -3 (stromelysin). J. Biol. Chem. 266, 15810-15816.
Mathis, D., Vence, L. & Benoist, C. (2001) Nature 414, 792-798.
Mohamadzadeh, M., DeGrendele, H., Arizpe, H., Estess, P. and Siegelman, M. (1998) Proinflammatory stimuli regulate endothelial hyaluronan expression and CD44/HA-dependent primary adhesion. J. Clin. Invest. 101, 97-108.
Mori, H., Tomari, T., Koshikawa, N., Kajita, M., Itoh, Y., Sato, H., Tojo, H., Yana, I. & Seiki, M. (2002) EMBO J. 21, 3949-3959.
Morimoto, Y., Nishikawa, K., and Ohashi, M. (1997) Life Sci. 61, 795-803
Nakamura, H., Suenaga, N., Taniwaki, K., Matsuki, H., Yonezawa, K., Fujii, M., Okada, Y. and Seiki, M. (2004) Constitutive and induced CD44 shedding by ADAM-like proteases and membrane-type 1 matrix metalloproteinase. Cancer Res. 64, 876-882.
Nandi, A., Estess, P. and Siegelman, M. (2004) Bimolecular complex between rolling and firm adhesion receptors required for cell arrest; CD44 association with VLA-4 in T cell extravasation. Immunity 20, 455-465.
Nguyen Q, Willenbrock F, Cockett M I, O“Shea M, Docherty A J & Murphy G (1994) Different domain interactions are involved in the binding of tissue inhibitors of metalloproteinases to stromelysin-1 and gelatinase A. Biochemistry 33: 2089-95.
O'Connell J P, Willenbrock F, Docherty A J, Eaton D & Murphy G (1994) Analysis of the role of the COOH-terminal domain in the activation, proteolytic activity, and tissue inhibitor of metalloproteinase interactions of gelatinase B. J Biol Chem 269: 14967-73.
O'Brien, P. M., Ortwine, D. F., Pavlovsky, A. G., Picard, J. A., Sliskovic, D. R., Roth, B. D., Dyer, R. D., Johnson, L. L., Man, C. F. and Hallak, H. (2000) Structure-activity relationships and pharmacokinetic analysis for a series of potent, systemically available biphenylsulfonamide matrix metalloproteinase inhibitors. J. Med. Chem. 43, 156-166.
Olson T M, Hirohata S, Ye J, Leco K, Seldin M F & Apte S S (1998) Cloning of the human tissue inhibitor of metalloproteinase-4 gene (TIMP4) and localization of the TIMP4 and Timp4 genes to human chromosome 3p25 and mouse chromosome 6, respectively. Genomics 51: 148-51.
Palosaari, H Acta Universitatis Ouluensis Medica. D 739, ISBN 951-42-7077-0.
Peterson, J. T., Hallak, H., Johnson, L., Li, H., O'Brien, P. M., Sliskovic, D. R., Bocan, T. M., Coker, M. L., Etoh, T. and Spinale, F. G. (2001) Matrix metalloproteinase inhibition attenuates left ventricular remodeling and dysfunction in a rat model of progressive heart failure. Circulation 103, 2303-2309.
Pomerleau, D. P., Bagley, R. J., Serreze, D. V., Mathews, C. E. & Leiter, E. H. (2005) Diabetes 54, 1603-1606.
Remacle, A. G., Rozanov, D. V., Fugere, M., Day, R. and Strongin, A. Y. (2006) Furin regulates the intracellular activation and the uptake rate of cell surface-associated MT1-MMP. Oncogene 25, 5648-5655.
Rifas L, Fausto A, Scott M J, Avioli L V & Welgus H G (1994) Expression of metalloproteinases and tissue inhibitors of metalloproteinases in human osteoblast-like cells: differentiation is associated with repression of metalloproteinase biosynthesis. Endocrinology 134: 213-21.
Rifas L, Halstead L R, Peck W A, Avioli L V & Welgus H G (1989) Human osteoblasts in vitro secrete tissue inhibitor of metalloproteinases and gelatinase but not interstitial collagenase as major cellular products. J Clin Invest 84: 686-94.
Rozanov, D. V., Deryugina, E. I., Ratnikov, B. I., Monosov, E. Z., Marchenko, G. N., Quigley, J. P., and Strongin, A. Y. (2001) J. Biol. Chem. 276, 25705-25714.
Ryan, M. E., Ramamurthy, N. S., Sorsa, T., and Golub, L. M. (1999) Ann. N. Y. Acad. Sci. 878, 311-334.
Sabeh, F., Ota, I., Holmbeck, K., Birkedal-Hansen, H., Soloway, P., Balbin, M., Lopez-Otin, C., Shapiro, S., Inada, M., Krane, S., Allen, E., Chung, D. & Weiss, S. J. (2004) J. Cell Biol. 167, 769-781.
Savinov, A. Y., Rozanov, D. V. and Strongin, A. Y. (2006) Mechanistic insights into targeting T cell membrane proteinase to promote islet beta-cell rejuvenation in type 1 diabetes. FASEB J. 20, 1793-1801.
Savinov, A. Y., Rozanov, D. V., Golubkov, V. S., Wong, F. S. and Strongin, A. Y. (2005) Inhibition of membrane type-1 matrix metalloproteinase by cancer drugs interferes with the homing of diabetogenic T cells into the pancreas. J. Biol. Chem. 280, 27755-27758.
Savinov, A. Y., Wong, F. S. & Chervonsky, A. V. (2001) J. Immunol. 167, 6637-6643.
Savinov, A. Y., Wong, F. S., Stonebraker, A. C. and Chervonsky, A. V. (2003) Presentation of antigen by endothelial cells and chemoattraction are required for homing of insulin-specific CD8+ T cells. J. Exp. Med. 197, 643-656.
Seiki, M. (2003) Cancer Lett. 194, 1-11.
Serreze, D. V., Leiter, E. H., Christianson, G. J., Greiner, D. and Roopenian, D. C. (1994) Major histocompatibility complex class I-deficient NOD-B2mnull mice are diabetes and insulitis resistant. Diabetes 43, 505-509.
Shalinsky, D. R., Brekken, J., Zou, H., McDermott, C. D., Forsyth, P., Edwards, D., Margosiak, S., Bender, S., Truitt, G., Wood, A., Varki, N. M. and Appelt, K. (1999) Broad antitumor and antiangiogenic activities of AG3340, a potent and selective MMP inhibitor undergoing advanced oncology clinical trials. Ann. N. Y. Acad. Sci. 878, 236-270.
Slevin, M., Kumar, S. and Gaffney, J. (2002) Angiogenic oligosaccharides of hyaluronan induce multiple signaling pathways affecting vascular endothelial cell mitogenic and wound healing responses. J. Biol. Chem. 277, 41046-41059.
Stetler-Stevenson W G, Krutzsch H C & Liotta L A (1989a) Tissue inhibitor of metalloproteinase (TIMP-2). A new member of the metalloproteinase inhibitor family. J Biol Chem 264: 17374-8.
Stratmann B, Farr M & Tschesche H (2001) Characterization of C-terminally truncated human tissue inhibitor of metalloproteinases-4 expressed in Pichia pastoris. Biol Chem 382: 987-91.
Strongin, A. Y. (2006) Mislocalization and unconventional functions of cellular MMPs in cancer. Cancer Metastasis Rev. 25, 87-98.
Strongin, A. Y., Collier, I., Bannikov, G., Marmer, B. L., Grant, G. A. and Goldberg, G. I. (1995) Mechanism of cell surface activation of 72-kDa type IV collagenase. Isolation of the activated form of the membrane metalloprotease. J. Biol. Chem. 270, 5331-5338.
Su S, Dehnade F & Zafarullah M (1996) Regulation of tissue inhibitor of metalloproteinases-3 gene expression by transforming growth factor-beta and dexamethasone in bovine and human articular chondrocytes. DNA Cell Biol 15: 1039-48.
Suenaga, N., Mori, H., Itoh, Y. and Seiki, M. (2005) CD44 binding through the hemopexin-like domain is critical for its shedding by membrane-type 1 matrix metalloproteinase. Oncogene 24, 859-868.
Traisman, H. S. & Greenwood, R. D. (1973) Clin. Pediatr. 12, 357-359.
Trim J E, Samra S K, Arthur M J, Wright M C, McAulay M, Beri R & Mann D A (2000) Upstream tissue inhibitor of metalloproteinases-1 (TIMP-1) element-1, a novel and essential regulatory DNA motif in the human TIMP-1 gene promoter, directly interacts with a 30-kDa nuclear protein. J Biol Chem 275: 6657-63.
Weber, C. (2003) Novel mechanistic concepts for the control of leukocyte transmigration: specialization of integrins, chemokines, and junctional molecules. J. Mol. Med. 81, 4-19.
Weiss, L., Slavin, S., Reich, S., Cohen, P., Shuster, S., Stem, R., Kaganovsky, E., Okon, E., Rubinstein, A. M. and Naor, D. (2000) Induction of resistance to diabetes in non-obese diabetic mice by targeting CD44 with a specific monoclonal antibody. Proc. Natl. Acad. Sci. USA 97, 285-290.
Wick M, Haronen R, Mumberg D, Burger C, Olsen B R, Budarf M L, Apte S S & Muller R (1995) Structure of the human TIMP-3 gene and its cell cycle-regulated promoter. Biochem J 311: 549-54.
Wilde C G, Hawkins P R, Coleman R T, Levine W B, Delegeane A M, Okamoto P M, Ito L Y, Scott R W & Seilhamer J J (1994) Cloning and characterization of human tissue inhibitor of metalloproteinases-3. DNA Cell Biol 13: 711-8.
Will, H., Atkinson, S. J., Butler, G. S., Smith, B. & Murphy, G. (1996) J. Biol. Chem. 271, 17119-17123.
Wong, F. S. and Janeway, C. A., Jr. (1999) The role of CD4 vs. CD8 T cells in IDDM. J. Autoimmun. 13, 290-295.
Wong, F. S., Karttunen, J., Dumont, C., Wen, L., Visintin, I., Pilip, I. M., Shastri, N., Pamer, E. G. & Janeway, C. A., Jr. (1999) Nat. Med. 5, 1026-1031.
Wong, F. S., Visintin, I., Wen, L., Flavell, R. A. and Janeway, C. A., Jr. (1996) CD8 T cell clones from young nonobese diabetic (NOD) islets can transfer rapid onset of diabetes in NOD mice in the absence of CD4 cells. J. Exp. Med. 183, 67-76.
Zafarullah M, Su S, Martel-Pelletier J, DiBattista J A, Costello B G, Stetler-Stevenson W G & Pelletier J P (1996) Tissue inhibitor of metalloproteinase-2 (TIMP-2) mRNA is constitutively expressed in bovine, human normal, and osteoarthritic articular chondrocytes. J Cell Biochem 60: 211-7.
Zhou, Y. P., Madjidi, A., Wilson, M. E., Nothhelfer, D. A., Johnson, J. H., Palma, J. F., Schweitzer, A., Burant, C., Blume, J. E. and Johnson, J. D. (2005) Matrix metalloproteinases contribute to insulin insufficiency in Zucker diabetic fatty rats. Diabetes 54, 2612-2619.
Zucker, S., Cao, J. & Chen, W. T. (2000) Oncogene 19, 6642-6650.
Zucker, S., Pei, D., Cao, J. and Lopez-Otin, C. (2003) Membrane type-matrix metalloproteinases (MT-MMP). Curr. Top. Dev. Biol. 54, 1-74.

### F. SEQUENCES

1. SEQ ID NO:1
lylvcgerg
2. SEQ ID NO:2 (TIMP-2)
mgaaartlrl algllllatl lrpadacscs pvhpqqafcn
advvirakav sekevdsgnd iygnpikriq yeikqikmfk
gpekdiefiy tapssavcgv sldvggkkey liagkaegdg
kmhitlcdfi vpwdtlsttq kkslnhryqm gceckitrcp
mipcyisspd eclwmdwvte kninghqakf facikrsdgs
cawyrgaapp kqefldiedp
3. SEQ ID NO:3 (TIMP-2)
agcaaacaca tccgtagaag gcagcgcggc cgccgagagc
cgcagcgccg ctcgcccgcc gccccccacc ccgccgcccc
gcccggcgaa ttgcgccccg cgcccctccc ctcgcgcccc
cgagacaaag aggagagaaa gtttgcgcgg ccgagcgggg
caggtgagga gggtgagccg cgcgggaggg gcccgcctcg
gccccggctc agcccccgcc cgcgccccca gcccgccgcc
gcgagcagcg cccggacccc ccagcggcgg cccccgcccg
cccagccccc cggcccgcca tgggcgccgc ggcccgcacc
ctgcggctgg cgctcggcct cctgctgctg gcgacgctgc
ttcgcccggc cgacgcctgc agctgctccc cggtgcaccc
gcaacaggcg ttttgcaatg cagatgtagt gatcagggcc
aaagcggtca gtgagaagga agtggactct ggaaacgaca
tttatggcaa ccctatcaag aggatccagt atgagatcaa
gcagataaag atgttcaaag ggcctgagaa ggatatagag
tttatctaca cggccccctc ctcggcagtg tgtggggtct
cgctggacgt tggaggaaag aaggaatatc tcattgcagg
aaaggccgag ggggacggca agatgcacat caccctctgt
gacttcatcg tgccctggga caccctgagc accacccaga
agaagagcct gaaccacagg taccagatgg gctgcgagtg
caagatcacg cgctgcccca tgatcccgtg ctacatctcc
tccccggacg agtgcctctg gatggactgg gtcacagaga
agaacatcaa cgggcaccag gccaagttct tcgcctgcat
caagagaagt gacggctcct gtgcgtggta ccgcggcgcg
gcgcccccca agcaggagtt tctcgacatc gaggacccat
aagcaggcct ccaacgcccc tgtggccaac tgcaaaaaaa
gcctccaagg gtttcgactg gtccagctct gacatccctt
cctggaaaca gcatgaataa aacactcatc ccatgggtcc
aaattaatat gattctgctc cccccttctc cttttagaca
tggttgtggg tctggaggga gacgtgggtc caaggtcctc
atcccatcct ccctctgcca ggcactatgt gtctggggct
tcgatccttg ggtgcaggca gggctgggac acgcggcttc
cctcccagtc cctgccttgg caccgtcaca gatgccaagc
aggcagcact tagggatctc ccagctgggt tagggcaggg
cctggaaatg tgcattttgc agaaactttt gagggtcgtt
gcaagactgt gtagcaggcc taccaggtcc ctttcatctt
gagagggaca tggcccttgt tttctgcagc ttccacgcct
ctgcactccc tgcccctggc aagtgctccc atcgccccgg
tgcccaccat gagctcccag cacctgactc cccccacatc
caagggcagc ctggaaccag tggctagttc ttgaaggagc
cccatcaatc ctattaatcc tcagaattcc agtgggagcc
tccctctgag ccttgtagaa atgggagcga gaaaccccag
ctgagctgcg ttccagcctc agctgagtct ttttggtctg
cacccacccc cccacccccc ccccgcccac atgctcccca
gcttgcagga ggaatcggtg aggtcctgtc ctgaggctgc
tgtccggggc cggtggctgc cctcaaggtc ccttccctag
ctgctgcggt tgccattgct tcttgcctgt tctggcatca
ggcacctgga ttgagttgca cagctttgct ttatccgggc
ttgtgtgcag ggcccggctg ggctccccat ctgcacatcc
tgaggacaga aaaagctggg tcttgctgtg ccctcccagg
cttagtgttc cctccctcaa agactgacag ccatcgttct
gcacggggtt ttctgcatgt gacgccagct aagcatagta
agaagtccag cctaggaagg gaaggatttt ggaggtaggt
ggctttggtg acacactcac ttctttctca gcctccagga
cactatggcc tgttttaaga gacatcttat ttttctaaag
gtgaattctc agatgatagg tgaacctgag ttgcagatat
accaacttct gcttgtattt cttaaatgac aaagattacc
tagctaagaa acttcctagg gaactaggga acctatgtgt
tccctcagtg tggtttcctg aagccagtga tatgggggtt
aggataggaa gaactttctc ggtaatgata aggagaatct
cttgtttcct cccacctgtg ttgtaaagat aaactgacga
tatacaggca cattatgtaa acatacacac gcaatgaaac
cgaagcttgg cggcctgggc gtggtcttgc aaaatgcttc
caaagccacc ttagcctgtt ctattcagcg gcaaccccaa
agcacctgtt aagactcctg acccccaagt ggcatgcagc
ccccatgccc accgggacct ggtcagcaca gatcttgatg
acttcccttt ctagggcaga ctgggagggt atccaggaat
cggcccctgc cccacgggcg ttttcatgct gtacagtgac
ctaaagttgg taagatgtca taatggacca gtccatgtga
tttcagtata tacaactcca ccagacccct ccaacccata
taacacccca cccctgttcg cttcctgtat ggtgatatca
tatgtaacat ttactcctgt ttctgctgat tgttttttta
atgttttggt ttgtttttga catcagctgt aatcattcct
gtgctgtgtt ttttattacc cttggtaggt attagacttg
cactttttta aaaaaaggtt tctgcatcgt ggaagcattt
gacccagagt ggaacgcgtg gcctatgcag gtggattcct
tcaggtcttt cctttggttc tttgagcatc tttgctttca
ttcgtctccc gtctttggtt ctccagttca aattattgca
aagtaaagga tctttgagta ggttcggtct gaaaggtgtg
gcctttatat ttgatccaca cacgttggtc ttttaaccgt
gctgagcaga aaacaaaaca ggttaagaag agccgggtgg
cagctgacag aggaagccgc tcaaatacct tcacaataaa
tagtggcaat atatatatag tttaagaagg ctctccattt
ggcatcgttt aatttatatg ttatgttcta agcacagctc
tcttctccta ttttcatcct gcaagcaact caaaatattt
aaaataaagt ttacattgta gttattttca aatctttgct
tgataagtat taagaaatat tggacttgct gccgtaattt
aaagctctgt tgattttgtt tccgtttgga tttttggggg
aggggagcac tgtgtttatg ctggaatatg aagtctgaga
ccttccggtg ctgggaacac acaagagttg ttgaaagttg
acaagcagac tgcgcatgtc tctgatgctt tgtatcattc
ttgagcaatc gctcggtccg tggacaataa acagtattat
caaagagaaa aaaaaaaaaa a
4. SEQ ID NO:4 (TIMP-3)
mtpwlglivl lgswslgdwg aeactcspsh pqdafcnsdi
virakvvgkk lvkegpfgtl vytikqmkmy rgftkmphvq
yihteasesl cglklevnky qylltgrvyd gkmytglcnf
verwdqltls qrkglnyryh lgcnckiksc yylpcfvtsk
neclwtdmls nfgypgyqsk hyacirqkgg ycswyrgwap
pdksiinatd p
5. SEQ ID NO:5 (TIMP-3)
cccgccggcg gcgcgcacgg caactttgga gaggcgagca
gcagccccgg cagcggcggc agcagcggca atgacccctt
ggctcgggct catcgtgctc ctgggcagct ggagcctggg
ggactggggc gccgaggcgt gcacatgctc gcccagccac
ccccaggacg ccttctgcaa ctccgacatc gtgatccggg
ccaaggtggt ggggaagaag ctggtaaagg aggggccctt
cggcacgctg gtctacacca tcaagcagat gaagatgtac
cgaggcttca ccaagatgcc ccatgtgcag tacatccaca
cggaagcttc cgagagtctc tgtggcctta agctggaggt
caacaagtac cagtacctgc tgacaggtcg cgtctatgat
ggcaagatgt acacggggct gtgcaacttc gtggagaggt
gggaccagct caccctctcc cagcgcaagg ggctgaacta
tcggtatcac ctgggttgta actgcaagat caagtcctgc
tactacctgc cttgctttgt gacttccaag aacgagtgtc
tctggaccga catgctctcc aatttcggtt accctggcta
ccagtccaaa cactacgcct gcatccggca gaagggcggc
tactgcagct ggtaccgagg atgggccccc ccggataaaa
gcatcatcaa tgccacagac ccctgagcgc cagaccctgc
cccacctcac ttccctccct tcccgctgag cttcccttgg
acactaactc ttcccagatg atgacaatga aattagtgcc
tgttttcttg caaatttagc acttggaaca tttaaagaaa
ggtctatgct gtcatatggg gtttattggg aactatcctc
ctggccccac cctgcccctt ctttttggtt ttgacatcat
tcatttccac ctgggaattt ctggtgccat gccagaaaga
atgaggaacc tgtattcctc ttcttcgtga taatataatc
tctatttttt taggaaaaaa a
6. SEQ ID NO:6 (TIMP-4)
mpgsprpaps wvlllrllal lrppglgeac scapahpqqh
ichsalvira kissekvvpa sadpadtekm lryeikqikm
fkgfekvkdv qyiytpfdss lcgvkleans qkqylltgqv
lsdgkvfihl cnyiepwedl slvqreslnh hyhlncgcqi
ttcytvpcti sapneclwtd wllerklygy qaqhyvcmkh
vdgtcswyrg hlplrkefvd ivqp
7. SEQ ID NO:7 (TIMP-4)
atcccctctc ccagtgcttc ccctctgctt ccagatcgct
tcatgactta ggcagggaaa cagaggtcag ggcctccttc
caggcttccc tctgcatctt actgagtatg caggtcggaa
gagcctcggg tcctgcctcc gcgggtggcc tagagccaaa
ggaaggcgga gcccgtcggg gcgggattgg cccttagggc
cacctcataa agcctggggc gaggggcaca acggccttgg
gaaggagccc tgctggggcc gtccagtccc ccagacctca
caggctcagt cgcggatctg cagtgtcatg cctgggagcc
ctcggcccgc gccaagctgg gtgctgttgc tgcggctgct
ggcgttgctg cggcccccgg ggctgggtga ggcatgcagc
tgcgccccgg cgcaccctca gcagcacatc tgccactcgg
cacttgtgat tcgggccaaa atctccagtg agaaggtagt
tccggccagt gcagaccctg ctgacactga aaaaatgctc
cggtatgaaa tcaaacagat aaagatgttc aaagggtttg
agaaagtcaa ggatgttcag tatatctata cgccttttga
ctcttccctc tgtggtgtga aactagaagc caacagccag
aagcagtatc tcttgactgg tcaggtcctc agtgatggaa
aagtcttcat ccatctgtgc aactacatcg agccctggga
ggacctgtcc ttggtgcaga gggaaagtct gaatcatcac
taccatctga actgtggctg ccaaatcacc acctgctaca
cagtaccctg taccatctcg gcccctaacg agtgcctctg
gacagactgg ctgttggaac gaaagctcta tggttaccag
gctcagcatt atgtctgtat gaagcatgtt gacggcacct
gcagctggta ccggggccac ctgcctctca ggaaggagtt
tgttgacatc gttcagccct agtagggacc agtgaccatc
acatcccttc aagagtcctg aagatcaagc cagttctcct
tccctgcaga gctttggcca ttaccacctg acctcttgct
gccagctaat aagaagtgcc aagtggacag tctggccact
gtcaaggcag ggaaggggcc atgacttttc tgccctgccc
tcagcctgtt gcccctgcct cccaaacccc attagtctag
ccttgtagct gttactgcaa gtgtttcttc tggcttagtc
tgttttctaa agccaggact attccctttc ctccccagga
atatgtgttt tcctttgtct taatcgatct ggtaggggag
aaatggcgaa tgtcatacac atgagatggt atatccttgc
gatgtacagt atcagaaggt ggtttgacag catcataaac
aggctgactg gcaggaatga aaacaagaaa aaaaaaaaaa
aaaaaaaaaa aaaaaaaaaa aaa

We claim:

1. A method of inhibiting the transmigration of T cells through pancreatic capillary endothelium, the method comprising administering to the cells a composition comprising an inhibitor of membrane type matrix metalloproteinase (MT-MMP).

2. The method of claim 1, wherein the MT-MMP is membrane type-1 matrix metalloproteinase (MT1-MMP).

3. The method of claim 1, wherein the cells are in or from a subject in need of inhibition of transmigration of T cells through pancreatic capillary endothelium.

4. The method of claim 1, wherein the inhibitor is a native tissue inhibitor of MMP (TIMP) or a hydoxamate.

5. The method of claim 4, wherein the hydroxamate is selected from the group consisting of BB-94, BB-1101, BB25-16, SE205, AG3340, and CGS 27023A.

6. The method of claim 4, wherein the hydroxamate is AG3340.

7. The method of claim 4, wherein the TIMP is selected from the group consisting of TIMP-2, TIMP-3, and TIMP-4.

8. The method of claim 4, wherein the TIMP is TIMP-2.

9. The method of claim 1, wherein the method results in an increase in T cell immobilization on the capillary endothelium surrounding pancreatic islet.

10. The method of claim 1, wherein the method results in a reduction in T cell homing.

11. The method of claim 1, wherein the inhibitor promotes regeneration of functional islets.

12. The method of claim 1, wherein the T cell is an insulin-specific, CD8-positive T cell.

13. The method of claim 1, wherein the inhibitor substantially immobilizes the T cells on the islet endothelium.

14. A method of treating type I diabetes in a subject, the method comprising administering to the subject a composition comprising an inhibitor of membrane type matrix metalloproteinase (MT-MMP).

15. The method of claim 14, wherein the MT-MMP is membrane type-1 matrix metalloproteinase (MT1-MMP).

16. The method of claim 14, wherein the subject is diagnosed as having type I diabetes.

17. The method of claim 14, wherein the inhibitor is an endogenous tissue inhibitor of MMP (TIMP) or a hydoxamate

18. The method of claim 17, wherein the hydroxamate is selected from the group consisting of BB-94, BB-1101, BB25-16, SE205, AG3340, and CGS 27023A.

19. The method of claim 17, wherein the hydroxamate is AG3340.

20. The method of claim 17, wherein the TIMP is selected from the group consisting of TIMP-2, TIMP-3, and TIMP-4.

21. The method of claim 17, wherein the TIMP is selected from the group consisting of TIMP-2, TIMP-3, and TIMP-4.

22. The method of claim 17, wherein the TIMP is TIMP-2.

23. The method of claim 14, wherein the inhibitor immobilizes the T cells on the islet endothelium.

24. The method of claim 14, wherein the inhibitor promotes regeneration of functional islets.

25. A method of identifying a molecule, the method comprising screening a candidate molecule for the ability to inhibit MT1-MMP activity, and determining if the candidate molecule can inhibit the transmigration of T cells through pancreatic capillary endothelium.

26. A method of identifying a molecule, the method comprising determining if a molecule that inhibits MT1-MMP activity can inhibit the transmigration of T cells through pancreatic capillary endothelium.

27. A method of immobilizing T cells on pancreatic capillary endothelium, the method comprising contacting the cells with a composition comprising an inhibitor of membrane type matrix metalloproteinase (MT-MMP).

28. The method of claim 27, wherein the MT-MMP is membrane type-I matrix metalloproteinase (MT1-MMP).

29. The method of claim 27, wherein the cells are in or from a subject identified as a subject in need of immobilization of T cells on pancreatic capillary endothelium.

30. A method of treating a subject at risk of type I diabetes, the method comprising administering to the subject a composition comprising an inhibitor of membrane type matrix metalloproteinase (MT-MMP).

31. The method of claim 30, wherein the MT-MMP is membrane type-I matrix metalloproteinase (MT1-MMP).
